(19) United States
(12) Patent Application Publication
(10) Pub. No.: US 2022/0042033 A1
(43) Pub. Date: Feb. 10, 2022

(54) COMPOSITION AND METHOD FOR CONFERRING AND/OR ENHANCING TOLERANCE AGAINST HERBICIDES BY USING VARIANTS OF PPO

(71) Applicant: FarmHannong Co., Ltd., Seoul (KR)

(72) Inventors: Soon-Kee SUNG, Daejeon (KR); Joonseon YOON, Daejeon (KR); Myoung-Ki HONG, Daejeon (KR); Young Ock AHN, Daejeon (KR); Joo Yong WOO, Daejeon (KR); Yunjung HAN, Daejeon (KR); Joonghyuk PARK, Daejeon (KR)

(21) Appl. No.: 16/772,928

(22) PCT Filed: Dec. 11, 2018
(86) PCT No.: PCT/KR2018/015654

§ 371 Date: Jun. 15, 2020

(30) Foreign Application Priority Data
Dec. 15, 2017 (KR) 10-2017-0173634

(51) Int. Cl. C12N 15/82; A01N 63/50; C07K 14/195; C12N 9/02; C12N 1/12

(52) U.S. Cl. CPC C12N 15/8274; A01N 63/50; C12Y 103/03004; C12N 9/001; C12N 1/12; C07K 14/195

(57) ABSTRACT

Provided is a technology for conferring more enhanced tolerance of plants and/or algae against herbicides and/or more greatly enhancing tolerance by using amino acid variants of protoporphyrinogen IX oxidases derived from microorganisms.

### TECHNICAL FIELD

Provided are PPO variants of a protoporphyrinogen IX oxidase for conferring and/or enhancing herbicide tolerance of a plant and/or algae using the same.

### BACKGROUND ART

A porphyrin biosynthetic pathway serves for the synthesis of chlorophyll and heme which play vital roles in plant metabolism, and it takes place in the chloroplast. In this pathway, protoporphyrinogen IX oxidase (hereinafter, referred to as PPO; EC:1.3.3.4) catalyzes the oxidation of protoporphyrinogen IX to protoporphyrin IX. After the oxidation of protoporphyrinogen IX to protoporphyrin IX, protoporphyrin IX binds with magnesium by Mg-chelatase to synthesize chlorophyll, or it binds with iron by Fe-chelatase to synthesize heme.

Therefore, when PPO activity is inhibited, synthesis of chlorophylls and heme is inhibited and the substrate protoporphyrinogen IX leaves the normal porphyrin biosynthetic pathway, resulting in the rapid export of protoporphyrinogen IX from the chloroplast to the cytoplasm, and cytoplasmic accumulation of protoporphyrin IX oxidized by nonspecific peroxidases and auto-oxidation. Accumulated protoporphyrin IX generates highly reactive singlet oxygen (¹O₂) in the presence of light and oxygen molecules which destroy cell membrane and rapidly leads to plant cell death. Based on this principle, herbicides inhibiting PPO activity have been developed. Until now, there have been 10 families of PPO-inhibiting herbicides, including pyrimidinediones, diphenyl-ethers, phenylpyrazoles, N-phenylphthalimides, thiadiazoles, oxadiazoles, triazinone, triazolinones, oxazolidinediones, and others herbicides, which are classified according to their chemical structures.

Further, in order to prevent effects of these herbicides on the growth of crops while using the herbicides, there is a need to provide herbicide tolerance for the crops.

Meanwhile, algae are photosynthetic organisms that can convert light energy into chemical energy which can be used to synthesize various useful compounds. For example, algae can fix carbon by photosynthesis and convert carbon dioxide into sugar, starch, lipids, fats, or other biomolecules, thereby removing greenhouse gases from the atmosphere. In addition, large-scale cultivation of algae can produce a variety of substances such as industrial enzymes, therapeutic compounds and proteins, nutrients, commercial materials and fuel materials.

However, in case of large-scale cultivation of algae in a bioreactor or in an open or enclosed pond, contamination may occur by undesired competent organisms, for example, undesired algae, fungi, rotifer, or zooplankton.

Thus, a technology is needed to harvest desired plants and/or algae on a large scale by treating herbicides at a concentration that would inhibit the growth of competent organisms without herbicide tolerance, after conferring herbicide tolerance to desired plants and/or algae.

### REFERENCES

(Patent document 1) U.S. Pat. No. 6,308,458 (2001 Oct. 30)

(Patent document 2) U.S. Pat. No. 6,808,904 (2004 Oct. 26)

(Patent document 3) U.S. Pat. No. 7,563,950 (2009 Jul. 21)

(Patent document 4) WO2011/085221 (2011 Jul. 14)

(Non-patent document 1) Li X, Volrath S L, Chilcott C E, Johnson M A, Ward E R, Law M D, Development of protoporphyrinogen IX oxidase as an efficient selection marker for Agrobacterium tumefaciens-mediated transformation of maize. Plant Physiol. 133:736-747, 2003

### DISCLOSURE

### Technical Problem

In this disclosure, it is found that hemY-type PPO genes derived from prokaryotes and mutants thereof show a broad herbicide tolerance to protoporphyrinogen IX oxidase (PPO)-inhibiting herbicides, thereby suggesting that the hemY-type PPO gene can confer and/or enhance herbicide tolerance when it is introduced in a plant and/or algae.

One embodiment provides a polypeptide variant comprising:

an amino acid sequence having modification to SEQ ID NO: 1, wherein the modification comprises deletion and/or substitution with a different amino acid from an original amino acid at one or more amino acids selected from amino acids involved in the interaction of a polypeptide of SEQ ID NO: 1 with a PPO-inhibiting herbicide (e.g., at least one amino acid selected from amino acids positioned on binding sites of the polypeptide of SEQ ID NO: 1 interacting with PPO-inhibiting herbicide), or

an amino acid sequence having at least 95%, at least 96%, at least 97%, at least 98%, or at least 99% identity with the amino acid sequence.

The at least one amino acid selected from the group consisting of amino acids of the polypeptide of SEQ ID NO: 1 involved in the interaction between PPO-inhibiting herbicides and the polypeptide, SEQ ID NO: 1, may be at least one amino acid selected from the group consisting of R140, F209, V213, A215, G216, V360, S362, F386, L389, L399, I402, and Y422, of the amino acid sequence of SEQ ID NO: 1.

Another embodiment provides a polypeptide variant the variant comprising: an amino acid sequence having modification to SEQ ID NO: 3, wherein the modification comprises deletion and/or substitution with a different amino acid from an original amino acid at one or more amino acids selected from amino acids involved in the interaction of a polypeptide of SEQ ID NO: 3 with a PPO-inhibiting herbicide (e.g., at least one amino acid selected from amino acids positioned on binding sites of the polypeptide of SEQ ID NO: 1 interacting with PPO-inhibiting herbicide), or

an amino acid sequence having at least 95%, at least 96%, at least 97%, at least 98%, or at least 99% sequence identity with the amino acid sequence.

The at least one amino acid selected from the group consisting of amino acids of the polypeptide of SEQ ID NO: 3 affecting to the interaction between PPO-inhibiting herbicides and the polypeptide, SEQ ID NO: 3, may be at least one amino acid selected from the group consisting of R95, V164, I168, A170, G171, I311, V313, F329, L332, L342, I345, and M365, of the amino acid sequence of SEQ ID NO: 3.

Another embodiment provides a polynucleotide encoding the polypeptide variant.

Another embodiment provides a recombinant vector comprising the polynucleotide.

Another embodiment provides a recombinant cell comprising the recombinant vector.

Another embodiment provides a composition for conferring and/or enhancing herbicide tolerance of a plant and/or algae, comprising at least one selected from the group consisting of:

a polypeptide variant having modification to SEQ ID NO: 1 or SEQ ID NO: 3, or a polypeptide comprising an amino acid sequence having 95% or higher, 96% or higher, 97% or higher, 98% or higher, or 99% or higher sequence identity with the polypeptide variant;

a polynucleotide encoding the polypeptide variant or the polypeptide comprising an amino acid sequence having 95% or higher, 96% or higher, 97% or higher, 98% or higher, or 99% or higher sequence identity with the polypeptide variant;

a recombinant vector comprising the polynucleotide; and

a recombinant cell comprising the recombinant vector.

In a concrete embodiment, the polynucleotide encoding the polypeptide of SEQ ID NO: 1 may comprise the nucleic acid sequence of SEQ ID NO: 7, the polynucleotide encoding the polypeptide of SEQ ID NO: 3 may comprise the nucleic acid sequence of SEQ ID NO: 4; but the polynucleotides may not be limited thereto.

The herbicide may be an herbicide inhibiting a protoporphyrinogen IX oxidase activity.

For example, the herbicide may be at least one selected from the group consisting of pyrimidinediones, diphenyl-ethers, phenylpyrazoles, N-phenylphthalimides, phenylesters, thiadiazoles, oxadiazoles, triazinone, triazolinones, oxazolidinediones, and other herbicides, but not be limited thereto.

In a specific embodiment, the herbicide may be at least one selected from the group consisting of tiafenacil, butafenacil, saflufenacil, benzfendizone, fomesafen, oxyfluorfen, aclonifen, acifluorfen, bifenox, ethoxyfen, lactofen, chlomethoxyfen, chlorintrofen, fluoroglycofen-ethyl, halosafen, pyraflufen-ethyl, fluazolate, flumioxazin, cinidon-ethyl, flumiclorac-pentyl, fluthiacet, thidiazimin, oxadiargyl, oxadiazon, carfentrazone, sulfentrazone, trifludimoxazin, azafenidin, pentoxazone, pyraclonil, flufenpyr-ethyl, profluazol, phenopylate (2,4-dichlorophenyl 1-pyrrolidinecarboxylate), carbamate analogues of phenopylate (for example, O-phenylpyrrolidino- and piperidinocarbamate analoges (refer to “Ujjana B. Nandihalli, Mary V. Duke, Stephen O. Duke, Relationships between molecular properties and biological activities of O-phenyl pyrrolidino- and piperidinocarbamate herbicides., J. Agric. Food Chem., 40(10) 1993-2000, 1992”)), agriculturally acceptable salts thereof, and combinations thereof, but not be limited thereto.

The plant may refer to a multicellular eukaryotic organism having photosynthetic capability, which may be a monocotyledonous plant or a dicotyledonous plant, or may be an herbaceous plant or a woody plant. The algae may refer to unicellular organism having photosynthetic capability, which may be prokaryotic algae or eukaryotic algae.

In an embodiment, the plant or algae may be genetically manipulated in order to further comprise a second herbicide tolerance polypeptide or a gene encoding the second herbicide tolerance polypeptide, whereby herbicide tolerance to the second herbicide can be conferred and/or enhanced. The plant or algae, which is genetically manipulated in order to comprise the second herbicide tolerance polypeptide or a gene encoding the second herbicide tolerance polypeptide, may be prepared using the second herbicide tolerance polypeptide or a gene encoding the second herbicide tolerance polypeptide in addition to the above mentioned composition for conferring and/or enhancing herbicide tolerance. Thus, a composition for conferring and/or enhancing tolerance to the herbicide may further comprise the second herbicide tolerance polypeptide or a gene encoding the second herbicide tolerance polypeptide.

Examples of the second herbicide may comprise cell division-inhibiting herbicides, photosynthesis-inhibiting herbicides, amino acid synthesis-inhibiting herbicides, plastid-inhibiting herbicides, cell membrane-inhibiting herbicides, and the like, but not be limited thereto.

In a specific embodiment, the second herbicide may be exemplified by glyphosate, glufosinate, dicamba, 2,4-D (2,4-Dichlorophenoxyacetic acid), isoxaflutole, ALS (acetolactate synthase)-inhibiting herbicide, photosystem II-inhibiting herbicide, or phenylurea-based herbicide, bromoxynil-based herbicide, or combinations thereof, but not be limited thereto.

For example, the second herbicide-tolerant polypeptide may be exemplified by at least one selected from the group consisting of glyphosate herbicide-tolerant EPSPS (glyphosate resistant 5-enolpyruvylshikimate-3-phosphate synthase), GOX (glyphosate oxidase), GAT (glyphosate-N-acetyltransferase) or glyphosate decarboxylase); glufosinate herbicide-tolerant PAT (phosphinothricin-N-acetyltransferase); dicamba herbicide-tolerant DMO (dicamba monooxygenase); 2,4-D herbicide-tolerant 2,4-D monooxygenase or AAD (aryloxyalkanoate dioxygenase); ALS-inhibiting sulfonylurea-based herbicide-tolerant ALS (acetolactate synthase), AHAS (acetohydroxyacid synthase), or AtAHASL (Arabidopsis thaliana acetohydroxyacid synthase large subunit); photosystem II-inhibiting herbicide-tolerant photosystem II protein D1; phenylurea-based herbicide-tolerant cytochrome P450; plastid-inhibiting herbicide-tolerant HPPD (hydroxyphenylpyruvate dioxygenase); bromoxynil herbicide-tolerant nitrilase; and combinations thereof, but not limited thereto.

In addition, the gene encoding the second herbicide-tolerant polypeptide may be exemplified by at least one selected from the group consisting of glyphosate herbicide-tolerant cp4 epsps, mepsps, 2mepsps, goxv247, gat4601 or gat4621 gene; glufosinate herbicide-tolerant bar, pat or pat (SYN) gene; dicamba herbicide-tolerant dmo gene; 2,4-D herbicide-tolerant AAD-1, AAD-12 gene; ALS-inhibiting sulfonylurea-based herbicide-tolerant ALS, GM-HRA, S4-HRA, ZM-HRA, Csr1, Csr1-1, Csr1-2, SurA or SurB; photosystem II-inhibiting herbicide-tolerant psbA gene; phenylurea herbicide-tolerant CYP76B1 gene; isoxaflutole herbicide-tolerant HPPDPF W336 gene and bromoxynil herbicide-tolerant bxn gene; and combinations thereof, but not limited thereto.

Another embodiment provides a transformant of a plant and/or algae having herbicide tolerance, which is transformed with the polynucleotide, or a clone or progeny thereof.

Another embodiment provides a method of preparing a transgenic plant or a transgenic alga having herbicide tolerance or enhanced herbicide tolerance, comprising a step of transforming a plant and/or algae with the polynucleotide.

Another embodiment provides a method of conferring or enhancing herbicide tolerance of a plant and/or algae, comprising a step of transforming a plant and/or algae with the polynucleotide.

The transformation may be performed to an alga, and/or a cell, protoplast, callus, hypocotyl, seed, cotyledon, shoot, or whole body of a plant.

The transformant may be an alga, and/or a cell, protoplast, callus, hypocotyl, seed, cotyledon, shoot, or whole body of a plant.

Another embodiment provides a method of controlling weeds in a cropland comprising:

providing a plant to the cropland, wherein the plant comprises at least one selected from the group consisting of the polypeptide, the variant of the polypeptide, a polynucleotide encoding the polypeptide, a polynucleotide encoding the variant, a recombinant vector comprising the polynucleotide, and a recombinant cell comprising the recombinant vector; and

applying an effective amount of a protoporphyrinogen IX oxidase-inhibiting herbicide to the cropland.

In a specific embodiment, the step of applying an effective amount of a protoporphyrinogen IX oxidase-inhibiting herbicide to the cropland may be performed by applying an effective amount of at least two protoporphyrinogen IX oxidase-inhibiting herbicides sequentially or simultaneously.

In another embodiment, the plant may be genetically manipulated in order to further comprise a second herbicide-tolerant polypeptide or a gene encoding the second herbicide-tolerant polypeptide, and an effective amount of the protoporphyrinogen IX oxidase-inhibiting herbicide and the second herbicide may be applied sequentially or simultaneously.

Another embodiment provides a method of removing an undesired organism from a culture medium, comprising providing an alga to a culture medium, wherein the algae comprises at least one selected from the group consisting of the polypeptide, the variant of the polypeptide, a polynucleotide encoding the polypeptide, a polynucleotide encoding the variant, a recombinant vector comprising the polynucleotide, and a recombinant cell comprising the recombinant vector; and applying an effective amount of a protoporphyrinogen IX oxidase-inhibiting herbicide to the culture medium.

### Technical Solution

Provided is a technology of conferring and/or enhancing herbicide tolerance of plants or algae.

As used herein, ‘conferring and/or enhancing herbicide tolerance of plants or algae’ or ‘enhancing herbicide tolerance of plants or algae’ may be interpreted as conferring herbicide tolerance to a plant or algae which do not have herbicide tolerance, and/or more strengthening herbicide tolerance of a plant or algae which have herbicide tolerance.

As used herein, ‘consisting of a sequence’ or ‘comprising a sequence’ may be used in order to cover both cases of comprising described sequence, and/or necessarily comprising the sequence, but it is not intended to exclude comprising further sequence other than the described sequence.

An embodiment provides a polypeptide variant which is at least one selected from the group consisting of:

a polypeptide variant comprising an amino acid sequence having modification to SEQ ID NO: 1, wherein the modification comprises deletion and/or substitution with a different amino acid from an original amino acid at one or more amino acids selected from amino acids involved in the interaction of a polypeptide of SEQ ID NO: 1 with a PPO-inhibiting herbicide (e.g., at least one amino acid selected from amino acids positioned on binding sites of the polypeptide of SEQ ID NO: 1 interacting with PPO-inhibiting herbicide), or an amino acid sequence having 95% or higher, 96% or higher, 97% or higher, 98% or higher, or 99% or higher sequence identity with the amino acid sequence; and

a polypeptide variant comprising an amino acid sequence having modification to SEQ ID NO: 3, wherein the modification comprises deletion and/or substitution with a different amino acid from an original amino acid at one or more amino acids selected from amino acids involved in the interaction of a polypeptide of SEQ ID NO: 3 with a PPO-inhibiting herbicide (e.g., at least one amino acid selected from amino acids positioned on binding sites of the polypeptide of SEQ ID NO: 3 interacting with PPO-inhibiting herbicide), or an amino acid sequence having 95% or higher, 96% or higher, 97% or higher, 98% or higher, or 99% or higher sequence identity with the amino acid sequence.

In other embodiment, provided is a polynucleotide encoding the polypeptide variant, a recombinant vector comprising the polynucleotide, and a recombinant cell comprising the recombinant vector. The polynucleotide may be designed in order to comprise a codon which is optimized to a cell to be transformed. The optimized codon may be easily known to a person skilled in the art (for example, refer to “http://www.genscript.com/codon-opt.html”, “http://sg.idtdna.com/CodonOpt”, etc.).

Another embodiment provides a composition for conferring and/or enhancing herbicide tolerance of a plant and/or algae, comprising at least one selected from the group consisting of:

a polypeptide variant having modification to SEQ ID NO: 1 or SEQ ID NO: 3, or a polypeptide comprising an amino acid sequence having 95% or higher, 96% or higher, 97% or higher, 98% or higher, or 99% or higher sequence identity with the polypeptide variant;

a polynucleotide encoding the polypeptide variant or the polypeptide comprising an amino acid sequence having 95% or higher, 96% or higher, 97% or higher, 98% or higher, or 99% or higher sequence identity with the polypeptide variant;

a recombinant vector comprising the polynucleotide; and

a recombinant cell comprising the recombinant vector.

In a concrete embodiment, the polynucleotide encoding the polypeptide of SEQ ID NO: 1 may comprise the nucleic acid sequence of SEQ ID NO: 7, the polynucleotide encoding the polypeptide of SEQ ID NO: 3 may comprise the nucleic acid sequence of SEQ ID NO: 4; but the polynucleotides may not be limited thereto.

In other embodiment, provided is a transformant of a plant and/or algae having herbicide tolerance, which is transformed with the polypeptide or a polynucleotide encoding the polypeptide. The polynucleotide may be designed in order to comprise a codon which is optimized to a cell to be transformed. The optimized codon may be easily known to a person skilled in the art (for example, refer to “http://www.genscript.com/codon-opt.html”, “http://sg.idtdna.com/CodonOpt”, etc.), etc.).

Another embodiment provides a method of preparing a transgenic plant or a transgenic algae having herbicide tolerance or enhanced herbicide tolerance, comprising a step of transforming a cell, protoplast, callus, hypocotyl, seed, cotyledon, shoot, or whole body of a plant or algae, with the polynucleotide.

Another embodiment provides a method of conferring or enhancing herbicide tolerance of a plant and/or algae, comprising a step of transforming a cell, protoplast, callus, hypocotyl, seed, cotyledon, shoot, or whole body of a plant or algae, with the polynucleotide.

The polypeptides of SEQ ID NO: 1 and 3 described herein are PPO proteins derived from a microorganism, and are herbicide-tolerant PPO proteins having tolerance to a PPO-inhibiting herbicide(s). Specifically, a PPO protein which is derived from Auxenochlorella protothecoides is provided, and it is designated as ApPPO1, and its amino acid sequence is represented by SEQ ID NO: 1. In addition, a PPO derived from Myxococcus xanthus is provided, and it is designated as MxPPO, and its amino acid sequence is represented by SEQ ID NO: 3, and a nucleotide sequence of a gene encoding the same is represented by SEQ ID NO: 4.

Herein, the polypeptide and variants of polypeptide may be expressed respectively as herbicide-tolerant PPO protein or herbicide-tolerant PPO protein variant having tolerance to a PPO-inhibiting herbicide(s). In addition, as used herein, the wording “a herbicide-tolerant PPO or its variant” may be used in order to refer to the above herbicide-tolerant PPO protein or herbicide-tolerant PPO protein variant, a herbicide-tolerant PPO protein-encoding gene or a herbicide-tolerant PPO protein variant-encoding gene, or all of them.

An amino acid mutation described herein may comprise substitution, deletion, addition and/or insertion at at least one amino acid selected from the amino acid residues of the interaction (binding) site of a PPO protein with a herbicide. Such a PPO protein having an amino acid mutation (that is, the polypeptide variant) may be one capable of maintaining the enzyme activity of the wild-type PPO protein.

The PPO protein variant will be described in more detail as follows.

One embodiment provides a polypeptide variant, which is a variant of a polypeptide of SEQ ID NO: 1 (ApPPO1), the variant comprising:

an amino acid sequence having modification to SEQ ID NO: 1 (ApPPO1), wherein the modification comprises deletion and/or substitution with a different amino acid from an original amino acid at one or more amino acids selected from amino acids involved in the interaction of a polypeptide of SEQ ID NO: 1 with a PPO-inhibiting herbicide (e.g., at least one amino acid selected from amino acids positioned on binding sites of the polypeptide of SEQ ID NO: 1 (ApPPO1) interacting with PPO-inhibiting herbicide), or an amino acid sequence having 95% or higher, 96% or higher, 97% or higher, 98% or higher, or 99% or higher sequence identity with the amino acid sequence; and The amino acid residue of SEQ ID NO: 1 to be deleted or substituted with other amino acid that is different from the original amino acid (e.g., at least one residue selected from the group consisting of amino acids positioned on binding sites to PPO-inhibiting herbicides of polypeptide of SEQ ID NO: 1) may be at least one selected from the group consisting of R140 (referring to “R(Arg) at the 140^{th} position; the expression of the following amino acid residues is interpreted in this manner), F209, V213, A215, G216, V360, S362, F386, L389, L399, I402, and Y422 of the amino acid sequence of SEQ ID NO: 1.

In one specific embodiment, the variant of polypeptide may comprise: an amino acid sequence having modification to SEQ ID NO: 1, wherein one or more amino acid residues selected from the group consisting of R140, F209, V213, A215, G216, V360, S362, F386, L389, L399, I402, and Y422 of the amino acid sequence of SEQ ID NO: 1 are respectively and independently deleted or substituted with an amino acid selected from the group consisting of M(Met), V(Val), I(Ile), T(Thr), L(Leu), C(Cys), A(Ala), S(Ser), F(Phe), P(Pro), W(Trp), N(Asn), Q(Gln), G(Gly), Y(Tyr), D(Asp), E(Glu), R(Arg), H(His), K(Lys), and the like, which is different from the amino acid at the corresponding position in the wild type (for example, one or more amino acid residues selected from the group consisting of R140, F209, V213, A215, G216, V360, S362, F386, L389, L399, I402, and Y422 of the amino acid sequence of SEQ ID NO: 1 are respectively and independently substituted with an amino acid selected from the group consisting of M(Met), V(Val), I(Ile), T(Thr), L(Leu), C(Cys), S(Ser), A(Ala), and the like, which is different from the amino acid at the corresponding position in the wild type), or

an amino acid sequence having at least 95%, at least 96%, at least 97%, at least 98%, or at least 99% sequence identity with the amino acid sequence.

For example, the variant of polypeptide may comprise:

an amino acid sequence having modification to SEQ ID NO: 1, wherein the modification comprises at least one amino acid mutation selected from the group consisting of Y422M (referring to a mutant or mutation wherein “the amino acid residue at the 422^{nd} position is substituted from Y(Tyre) to M(Met)”; the expression of the following amino acid mutations is interpreted in this manner), Y422L, Y422C, Y422V, Y422I, Y422T, A215L, A215C, A215I, V360M, R140A, F209A, V213C, V213S, F386V, L389T, I402T, V360I, V360L, and S362V, in the amino acid sequence of SEQ ID NO: 1; or an amino acid sequence having at least 95%, at least 96%, at least 97%, at least 98%, or at least 99% sequence identity with the amino acid sequence.

More specifically, the variant of polypeptide may comprise:

an amino acid sequence having modification to SEQ ID NO: 1, wherein the modification comprises at least one amino acid mutation selected from the group consisting of amino acid mutations of Y422M, Y422L, Y422C, Y422V, Y422I, Y422T, A215L, A215C, A215I, V360M, R140A, F209A, V213C, V213S, F386V, L389T, I402T, V360I, V360L, S362V, R140A+Y422I (referring to a mutant or mutation comprising all of substitution of the 140^{th} residue from R to A and substitution of the 422^{nd} residue from Y to I; the expression of the following two or more amino acid mutations is interpreted in this manner), R140A+Y422T, R140A+Y422M, F209A+Y422M, V213C+Y422I, V213C+Y422T, V213C+Y422M, A215C+Y422I, A215C+Y422T, A215C+Y422M, A215L+Y422I, A215L+Y422T, A215L+Y422M, V360M+Y422M, F386V+Y422M, V360M+Y422I, L389T+Y422M, I402T+Y422M, V360I+Y422I, V360I+S362V, S362V+Y422I, R140A+V213C+Y422I, R140A+V213C+Y422M, R140A+A215C+Y422I, R140A+A215L+Y422M, V213C+A215C+Y422I, V213C+A215L+Y422M, V360I+S362V+Y422I, A215C+V360M+Y422M, A215L+V360M+Y422M, A215I+V360M+Y422M, V213C+A215C+Y422M, V213C+A215L+Y422M, R140A+V213C+A215C+Y422I, or R140A+V213C+A215L+Y422M, in the amino acid sequence of SEQ ID NO: 1, or

an amino acid sequence having at least 95%, at least 96%, at least 97%, at least 98%, or at least 99% sequence identity with the amino acid sequence.

Another embodiment provides a polypeptide variant, which is a variant of a polypeptide of SEQ ID NO: 3 (MxPPO), the variant comprising:

an amino acid sequence having modification to SEQ ID NO: 3 (MxPPO), wherein the modification comprises deletion and/or substitution with a different amino acid from an original amino acid at one or more amino acids selected from amino acids involved in the interaction of a polypeptide of SEQ ID NO: 3 with a PPO-inhibiting herbicide (e.g., at least one amino acid selected from amino acids positioned on binding sites of the polypeptide of SEQ ID NO: 3 (MxPPO) interacting with PPO-inhibiting herbicide), or

an amino acid sequence having at least 95%, at least 96%, at least 97%, at least 98%, or at least 99% sequence identity with the amino acid sequence.

The amino acid residue of polypeptide of SEQ ID NO: 3 to be deleted or substituted with other amino acid which is different from the original amino acid (e.g., at least one residue selected from the group consisting of amino acids positioned on binding sites to PPO-inhibiting herbicides of polypeptide of SEQ ID NO: 3), may be at least one selected from the group consisting of R95, V164, I168, A170, G171, I311, V313, F329, L332, L342, I345, and M365 of the amino acid sequence of SEQ ID NO: 3.

In one specific embodiment, the variant of polypeptide may comprise:

an amino acid sequence having modification to SEQ ID NO: 3, wherein one or more amino acid residues selected from the group consisting of R95, V164, I168, A170, G171, I311, V313, F329, L332, L342, I345, and M365 of the amino acid sequence of SEQ ID NO: 3 are respectively and independently deleted or substituted with an amino acid selected from the group consisting of M(Met), V(Val), I(Ile), T(Thr), L(Leu), C(Cys), A(Ala), S(Ser), F(Phe), P(Pro), W(Trp), N(Asn), Q(Gln), G(Gly), Y(Tyr), D(Asp), E(Glu), R(Arg), H(His), K(Lys), and the like, which is different from the amino acid at the corresponding position in the wild type (for example, one or more amino acid residues selected from the group consisting of R95, V164, I168, A170, G171, I311, V313, F329, L332, L342, I345, and M365 of the amino acid sequence of SEQ ID NO: 3 are respectively and independently substituted with an amino acid selected from the group consisting of M(Met), V(Val), I(Ile), T(Thr), L(Leu), C(Cys), S(Ser), A(Ala), and the like, which is different from the amino acid at the corresponding position in the wild type), or

an amino acid sequence having at least 95%, at least 96%, at least 97%, at least 98%, or at least 99% sequence identity with the amino acid sequence.

For example, the variant of polypeptide may comprise:

an amino acid sequence having modification to SEQ ID NO: 3, wherein the modification comprises at least one amino acid mutation selected from the group consisting of M365T, M365L, M365C, M365V, M365I, R95A, V164A, I168C, I168S, A170C, A170L, A170I, I311M, F329V, L332T, and I345T, in the amino acid sequence of SEQ ID NO: 3; or an amino acid sequence having at least 95%, at least 96%, at least 97%, at least 98%, or at least 99% sequence identity with the amino acid sequence.

More specifically, the variant of polypeptide may comprise:

an amino acid sequence having modification to SEQ ID NO: 3, wherein the modification comprises at least one amino acid mutation selected from the group consisting of amino acid mutations of M365T, M365L, M365C, M365V, M365I, R95A, V164A, I168C, I168S, A170C, A170L, A170I, I311M, F329V, L332T, I345T, R95A+M365I, R95A+M365V, I168C+M365I, I168C+M365V, A170C+M365I, A170C+M365V, A170L+M365I, A170L+M365V, I311M+M365I, I311M+M365V, L332T+M365I, L332T+M365V, V164A+M365I, F329V+M365I, I345T+M365I, A170C+I311M, A170L+I311M, A170I+I311M, I168C+A170C, I168C+A170L, R95A+I168C+M365I, R95A+I168C+M365V, R95A+A170C+M365I, R95A+I311M+M365I, R95A+I311M+M365V, R95A+L332T+M365I, R95A+L332T+M365V, I168C+A170C+M365V, I168C+I311M+M365I, I168C+I311M+M365V, I168C+L332T+M365I, I168C+L332T+M365V, A170C+I311M+M365I, A170C+L332T+M365V, I311M+L332T+M365I, I311M+L332T+M365V, R95A+I168C+A170C+M365I, R95A+I168C+A170C+M365V, R95A+A170C+I311M+M365V, R95A+A170C+L332T+M365I, R95A+I168C+I311M+M365V, R95A+I168C+L332T+M365I, R95A+I311M+L332T+M365I, R95A+I311M+L332T+M365V, I168C+A170C+I311M+M365I, I168C+A170C+L332T+M365V, A170C+I311M+L332T+M365I, R95A+I168C+A170C+I311M+M365V, R95A+I168C+A170C+L332T+M365I, R95A+I168C+I311M+L332T+M365V, I168C+A170C+I311M+L332T+M365V, or R95A+I168C+A170C+I311M+L332T+M365V, in the amino acid sequence of SEQ ID NO: 3, or

an amino acid sequence having at least 95%, at least 96%, at least 97%, at least 98%, or at least 99% sequence identity with the amino acid sequence.

The polypeptide variant comprising an amino acid sequence having sequence identity (for example, 95% or higher, 98% or higher, or 99% or higher sequence identity) described herein may maintain enzyme activity equivalent to that of a polypeptide having an amino acid sequence which is a standard of identification of sequence identity (for example, the PPO protein having amino acid mutation described above), for example, 5% or higher, 10% or higher, 20% or higher, 30% or higher, 40% or higher, 50% or higher, 60% or higher, 70% or higher, 80% or higher, 90% or higher, or 95% or higher enzyme activity to a polypeptide having an amino acid sequence which is a standard in plants (in a whole plant, in a plant cell or cell culture, in a plant tissue, etc.), in algae, and/or in vitro, and having function to confer herbicide tolerance. The sequence identity description is used in order to clarify that the herbicide-tolerance PPO protein variant or polypeptide variant described herein may comprise any sequence mutation within the range capable of satisfying the above condition (maintaining enzymatic activity and possessing a function to confer herbicide tolerance).

The amino acids used in the description are summarized as follows:

| Amino acid | 3-letter code | 1-letter code |
|---|---|---|
| Alanine | Ala | A |
| Isoleucine | Ile | I |
| Leucine | Leu | L |
| Methionine | Met | M |
| Phenylalanine | Phe | F |
| Proline | Pro | P |
| Tryptophan | Trp | W |
| Valine | Val | V |
| Aspargine | Asn | N |
| Cysteine | Cys | C |
| Glutamine | Gln | Q |
| Glycine | Gly | G |
| Serine | Ser | S |
| Threonine | Thr | T |
| Tyrosine | Tyr | Y |
| Aspartic acid | Asp | D |
| Glutamic acid | Glu | E |
| Arginine | Arg | R |
| Histidine | His | H |
| Lysine | Lys | K |

The polypeptide variant (herbicide-tolerant PPO protein variant) may maintain its enzymatic activities as a PPO protein, and exhibit increased herbicide tolerance compared to the wild type.

In addition, the polypeptide variant (herbicide-tolerant PPO protein variant) may comprise further mutation exhibiting biologically equal activity to a polypeptide consisting of SEQ ID NO: 1, SEQ ID NO: 3, or an amino acid sequence having amino acid mutation(s) described above. For example, the additional mutation may be amino acid substitution which does not entirely alter molecular activity, and such amino acid substitution may be properly selected by a person skilled in the relevant art. In one example, the additional substitution may be substitution between amino acid residues Ala/Ser, Val/Ile, Asp/Glu, Thr/Ser, Ala/Gly, Ala/Thr, Ser/Asn, Ala/Val, Ser/Gly, Thr/Phe, Ala/Pro, Lys/Arg, Asp/Asn, Leu/Ile, Leu/Val, Ala/Glu, or Asp/Gly, but not be limited thereto. In some cases, the herbicide-tolerant PPO protein variant may be subjected to at least one modification selected from the group consisting of phosphorylation, sulfation, acylation, glycosylation, methylation, farnesylation, and the like. In addition, the herbicide-tolerant PPO protein variant may be one having increased structural stability to heat, pH, etc. of the protein, or increased protein activity by amino acid variation (mutation) and/or modification.

The term “sequence identity” refers to the degree of similarity to the wild type or reference amino acid sequence or nucleotide sequence, and any protein may be included in the scope of the present invention, as long as it includes amino acid residues having 60% or higher, 65% or higher, 70% or higher, 75% or higher, 80% or higher, 85% or higher, 90% or higher, 95% or higher, 98% or higher, or 99% or higher identity to the amino acid sequence of the herbicide-tolerant PPO protein variant as described above, and retains biological activities equivalent to the herbicide-tolerant PPO protein variant. Such protein homologues may comprise an active site equivalent to that of a targeted protein (the herbicide-tolerant PPO protein variant as described above).

The herbicide-tolerant PPO protein or its variant may be obtained by extracting and/or purifying from nature by methods well known in the relevant art. Alternatively, it may be obtained as a recombinant protein using a gene recombination technology. In case of using a gene recombination technology, it may be obtained by a process of introducing a nucleic acid encoding the herbicide-tolerant PPO protein or its variant into an appropriate expression vector, and introducing the expression vector into a host cell in order to express the herbicide-tolerant PPO protein or its variant, and then collecting the expressed herbicide-tolerant PPO protein or its variant from the host cell. After the protein is expressed in a selected host cell, the protein can be separated and/or purified by general biochemical separation techniques, for example, treatment with a protein precipitating agent (salting out), centrifugation, ultrasonic disruption, ultrafiltration, dialysis, chromatography such as molecular sieve chromatography (gel filtration), adsorption chromatography, ion exchange chromatography, affinity chromatography and the like, and in order to separate the protein with a high purity, these methods may be used in combination.

The herbicide-tolerant PPO nucleic acid molecule (polynucleotide encoding the PPO protein or its variant) may be isolated or prepared using standard molecular biological techniques, for example, a chemical synthesis or recombination method, or as the herbicide-tolerant PPO nucleic acid molecule, commercially available one can be used.

In this disclosure, the PPO proteins/nucleic acids or variants thereof were found to exhibit broad herbicide tolerance against representative 10 families of PPO inhibiting herbicides classified according to their chemical structures in a herbicide tolerance test system using PPO-deficient E. coli BT3(ΔPPO). It was also found that the proteins may be expressed in the chloroplast of a plant by using a transit peptide (TP). Further, it was found that the PPO proteins/nucleic acids or variants thereof may be also expressed in a monocotyledon, such as Oryza sativa, or a dicotyledon, such as, Arabidopsis thaliana ecotype Columbia-0 (A. thaliana), by a plant expression vector. Even when the transformed plants are treated with PPO-inhibiting herbicides, germination and growth of the plants are observed. Furthermore, it was confirmed, by an inheritance study, that the above herbicide-tolerant traits can be successfully inherited to the next generation.

Therefore, the PPO protein and its variants provided herein may be introduced into a plant or algae, thereby conferring herbicide tolerance to the plant or algae, and/or enhancing herbicide tolerance of the plant or algae.

One embodiment provides a composition for conferring and/or enhancing herbicide tolerance of plants and/or algae, comprising at least one selected from the group consisting of:

(1) a polypeptide variant as described above or comprising an amino acid sequence having at least 95%, at least 96%, at least 97%, at least 98%, or at least 99% sequence identity thereto;

(2) a polynucleotide encoding the polypeptide variant;

(3) a recombinant vector comprising the polynucleotide; and

(4) a recombinant cell comprising the recombinant vector.

The herbicide herein refers to an active ingredient that kills, controls, or otherwise adversely modifies the growth of plants or algae. In addition, the herbicide tolerance means that even after treatment of a herbicide which normally kills a normal or wild-type plant or normally inhibits growth thereof, inhibition of the plant growth is weakened or eliminated, compared to that of the normal or wild-type plant, and therefore, the plant continues to grow. The herbicide includes a herbicide inhibiting protoporphyrinogen IX oxidase (PPO) of a plant or an alga. Such PPO-inhibiting herbicide may be classified into pyrimidinediones, diphenyl-ethers, phenylpyrazoles, N-phenylphthalimides, phenylesters, thiadiazoles, oxadiazoles, triazolinones, oxazolidinediones, and other herbicides, according to their chemical structures.

As a specific embodiment, the pyrimidinedione-based herbicide may include butafenacil, saflufenacil, benzfendizone, and tiafenacil, but not be limited thereto.

The diphenyl-ether-based herbicide may include fomesafen, oxyfluorfen, aclonifen, acifluorfen, bifenox, ethoxyfen, lactofen, chlomethoxyfen, chlorintrofen, fluoroglycofen-ethyl, and halosafen, but not be limited thereto.

The phenylpyrazole-based herbicide may include pyraflufen-ethyl and fluazolate, but not be limited thereto.

The phenylphthalimide-based herbicide may include flumioxazin, cinidon-ethyl, and flumiclorac-pentyl, but not be limited thereto.

The phenylesters herbicide may include phenopylate (2,4-dichlorophenyl 1-pyrrolidinecarboxylate) and carbamate analogues of phenopylate (for example, O-phenylpyrrolidino- and piperidinocarbamate analoges (refer to “Ujjana B. Nandihalli, Mary V. Duke, Stephen O. Duke, Relationships between molecular properties and biological activities of O-phenyl pyrrolidino- and piperidinocarbamate herbicides., J. Agric. Food Chem., 40(10) 1993-2000, 1992”)), and the like, but not be limited thereto. In one specific embodiment, the carbamate analogue of phenopylate may be one or more selected from the group consisting of pyrrolidine-1-carboxylic acid phenyl ester (CAS No. 55379-71-0), 1-pyrrolidinecarboxylicacid, 2-chlorophenyl ester (CAS No. 143121-06-6), 4-chlorophenyl pyrrolidine-1-carboxylate (CAS No. 1759-02-0), carbamic acid, diethyl-,2,4-dichloro-5-(2-propynyloxy)phenyl ester (9CI) (CAS No. 143121-07-7), 1-pyrrolidinecarboxylicacid, 2,4-dichloro-5-hydroxyphenyl ester (CAS No. 143121-08-8), 2,4-dichloro-5-(methoxycarbonyl)phenyl pyrrolidine-1-carboxylate (CAS No. 133636-94-9), 2,4-dichloro-5-[(propan-2-yloxy)carbonyl]phenyl pyrrolidine-1-carboxylate (CAS No. 133636-96-1), 1-piperidinecarboxylic acid, 2,4-dichloro-5-(2-propynyloxy)phenyl ester (CAS No. 87374-78-5), 2,4-dichloro-5-(prop-2-yn-1-yloxy)phenyl pyrrolidine-1-carboxylate (CAS No. 87365-63-7), 2,4-dichloro-5-(prop-2-yn-1-yloxy)phenyl 4,4-difluoropiperidine-1-carboxylate (CAS No. 138926-22-4), 1-pyrrolidinecarboxylicacid, 3,3-difluoro-,2,4-dichloro-5-(2-propyn-1-yloxy)phenyl ester (CAS No. 143121-10-2), 4-chloro-2-fluoro-5-[(propan-2-yloxy)carbonyl]phenyl pyrrolidine-1-carboxylate (CAS No. 133636-98-3), and the like.

The thiadiazole-based herbicide may include fluthiacet and thidiazimin, but not be limited thereto.

The oxadiazole-based herbicide may include oxadiargyl and oxadiazon, but not be limited thereto.

The triazinone-based herbicide may include trifludimoxazin, but not be limited thereto.

The triazolinone-based herbicide may include carfentrazone, sulfentrazone, and azafenidin, but not be limited thereto.

The oxazolidinedione-based herbicide may include pentoxazone, but not be limited thereto.

The other herbicide may include pyraclonil, flufenpyr-ethyl, and profluazol, but not be limited thereto.

The herbicide-tolerant PPO gene provided herein may be introduced into a plant or algae by various methods known in the art, and preferably, by using an expression vector for plant or alga transformation.

In case of introducing the gene into a plant, an appropriate promoter which may be included in the vector may be any promoter generally used in the art for introduction of the gene into the plant. For example, the promoter may include an SP6 promoter, a T7 promoter, a T3 promoter, a PM promoter, a maize ubiquitin promoter, a cauliflower mosaic virus (CaMV) 35S promoter, a nopaline synthase (nos) promoter, a figwort mosaic virus 35S promoter, a sugarcane bacilliform virus promoter, a commelina yellow mottle virus promoter, a light-inducible promoter from the small subunit of ribulose-1,5-bisphosphate carboxylase (ssRUBISCO), a rice cytosolic triosephosphate isomerase (TPI) promoter, an adenine phosphoribosyltransferae (APRT) promoter of A. thaliana, an octopine synthase promoter, and a BCB (blue copper binding protein) promoter, but not be limited thereto.

Further, the vector may include a poly A signal sequence causing polyadenylation of 3′-terminus, and for example, it may include NOS 3′-end derived from a nopaline synthase gene of Agrobacterium tumefaciens, an octopine synthase terminator derived from an octopine synthase gene of Agrobacterium tumefaciens, 3′-end of protease inhibitor I or II gene of tomato or potato, a CaMV 35S terminator, a rice α-amylase terminator RAmyl A, and a phaseolin terminator, but not be limited thereto.

In addition, the case of introducing the gene into an alga, chloroplast-specific promoter, nucleus promoter, constitutive promoter, or inducible promoter may be used for introduction of the gene into the algae as a promoter. The herbicide-tolerant PPO gene or its variant provided herein may be designed in order to operationally link to 5′ UTR or 3′ UTR, thereby expressing function in nucleus of algae. In addition, the vector may further comprise a transcriptional regulatory sequence which is appropriate to transformation of algae. A recombinant gene conferring herbicide tolerance may be integrated to genome of nucleus or genome of chloroplast in a host alga, but not be limited thereto.

In addition, in the vector, a transit peptide required for targeting to chloroplasts may be linked to 5′-end of the PPO gene in order to express the herbicide-tolerant PPO gene in the chloroplasts.

In addition, optionally, the vector may further include a gene encoding selectable marker as a reporter molecule, and example of the selectable marker may include a gene having tolerance to an antibiotic (e.g., neomycin, carbenicillin, kanamycin, spectinomycin, hygromycin, bleomycin, chloramphenicol, ampicillin, etc.) or herbicide (glyphosate, glufosinate, phosphinothricin, etc.), but is not limited thereto.

Further, the recombinant vector for plant expression may include an Agrobacterium binary vector, a cointegration vector, or a general vector which has no T-DNA region but is designed to be expressed in the plant. Of them, the binary vector refers to a vector containing two separate vector systems harboring one plasmid responsible for migration consisting of left border (LB) and right border (RB) in Ti (tumor inducing) plasmid, and the other plasmid for target gene-transferring, and the vector may include a promoter region and a polyadenylation signal sequence for expression in plants.

When the binary vector or cointegration vector is used, a strain for transformation of the recombinant vector into the plant is preferably Agrobacterium (Agrobacterium-mediated transformation). For this transformation, Agrobacterium tumefaciens or Agrobacterium rhizogenes may be used. In addition, when the vector having no T-DNA region is used, electroporation, particle bombardment, polyethylene glycol-mediated uptake, and the like may be used for introduction of the recombinant plasmid into the plant.

The plant transformed with the gene by the above method may be re-differentiated into a plant through callus induction, rhizogenesis, and soil acclimatization, using a standard technique known in the relevant art.

The plant subjected to transformation herein may cover not only a mature plant but also a plant cell (containing a suspension-cultured cell), a protoplast, a callus, a hypocotyl, a seed, a cotyledon, a shoot, and the loke, which can grow to a mature plant.

Further, the scope of the transformant may include a transformant which the gene is introduced as well as a clone or progeny thereof (T₁ generation, T₂ generation, T₃ generation, T₄ generation, T₅ generation, or any subsequent generations). For example, the transformed plant also includes a plant having the inherited herbicide tolerance traits as sexual and asexual progeny of the plant transformed with the gene provided herein. The scope of the present invention also includes all mutants and variants showing the characteristics of the initial transformed plant, together with all hybridization and fusion products of the plant transformed with the gene provided herein. Furthermore, the scope of the present invention also includes a part of the plant, such as a seed, a flower, a stem, a fruit, a leaf, a root, a tuber, and/or a tuberous root, which is originated from a transformed plant which is transformed in advance by the method of the present invention, or a progeny thereof, and is composed of at least a part of the transformed cells.

The plant, to which the present invention is applied, is not particularly limited to, but may be at least one selected from the group consisting of monocotyledonous or dicotyledonous plants. Further, the plant may be at least one selected from the group consisting of herbaceous plants and woody plants. The monocotyledonous plant may include plants belonging to families Alismataceae, Hydrocharitaceae, Juncaginaceae, Scheuchzeriaceae, Potamogetonaceae, Najadaceae, Zosteraceae, Liliaceae, Haemodoraceae, Agavaceae, Amaryllidaceae, Dioscoreaceae, Pontederiaceae, Iridaceae, Burmanniaceae, Juncaceae, Commelinaceae, Eriocaulaceae, Gramineae (Poaceae), Araceae, Lemnaceae, Sparganiaceae, Typhaceae, Cyperaceae, Musaceae, Zingiberaceae, Cannaceae, Orchidaceae, and the like, but not be limited thereto.

The dicotyledonous plant may include plants belonging to families Diapensiaceae, Clethraceae, Pyrolaceae, Ericaceae, Myrsinaceae, Primulaceae, Plumbaginaceae, Ebenaceae, Styracaceae, Symplocaceae, Symplocaceae, Oleaceae, Loganiaceae, Gentianaceae, Menyanthaceae, Apocynaceae, Asclepiadaceae, Rubiaceae, Polemoniaceae, Convolvulaceae, Boraginaceae, Verbenaceae, Labiatae, Solanaceae, Scrophulariaceae, Bignoniaceae, Acanthaceae, Pedaliaceae, Orobanchaceae, Gesneriaceae, Lentibulariaceae, Phrymaceae, Plantaginaceae, Caprifoliaceae, Adoxaceae, Valerianaceae, Dipsacaceae, Campanulaceae, Compositae, Myricaceae, Juglandaceae, Salicaceae, Betulaceae, Fagaceae, Ulmaceae, Moraceae, Urticaceae, Santalaceae, Loranthaceae, Polygonaceae, Phytolaccaceae, Nyctaginaceae, Aizoaceae, Portulacaceae, Caryophyllaceae, Chenopodiaceae, Amaranthaceae, Cactaceae, Magnoliaceae, Illiciaceae, Lauraceae, Cercidiphyllaceae, Ranunculaceae, Berberidaceae, Lardizabalaceae, Menispermaceae, Nymphaeaceae, Ceratophyllaceae, Cabombaceae, Saururaceae, Piperaceae, Chloranthaceae, Aristolochiaceae, Actinidiaceae, Theaceae, Guttiferae, Droseraceae, Papaveraceae, Capparidaceae, Cruciferae, Platanaceae, Hamamelidaceae, Crassulaceae, Saxifragaceae, Eucommiaceae, Pittosporaceae, Rosaceae, Leguminosae, Oxalidaceae, Geraniaceae, Tropaeolaceae, Zygophyllaceae, Linaceae, Euphorbiaceae, Callitrichaceae, Rutaceae, Simaroubaceae, Meliaceae, Polygalaceae, Anacardiaceae, Aceraceae, Sapindaceae, Hippocastanaceae, Sabiaceae, Balsaminaceae, Aquifoliaceae, Celastraceae, Staphyleaceae, Buxaceae, Empetraceae, Rhamnaceae, Vitaceae, Elaeocarpaceae, Tiliaceae, Malvaceae, Sterculiaceae, Thymelaeaceae, Elaeagnaceae, Flacourtiaceae, Violaceae, Passifloraceae, Tamaricaceae, Elatinaceae, Begoniaceae, Cucurbitaceae, Lythraceae, Punicaceae, Onagraceae, Haloragaceae, Alangiaceae, Cornaceae, Araliaceae, Umbelliferae (Apiaceae)), and the like, but not be limited thereto.

In a specific embodiment, the plant may be at least one selected from the group consisting of food crops such as rice, wheat, barley, corn, soybean, potato, red bean, oat, and sorghum; vegetable crops such as Chinese cabbage, radish, red pepper, strawberry, tomato, watermelon, cucumber, cabbage, oriental melon, pumpkin, welsh anion, anion, and carrot; crops for special use such as ginseng, tobacco, cotton, soilage, forage, sesame, sugar cane, sugar beet, Perilla sp., peanut, rapeseed, grass, and castor-oil plant; fruit trees such as apple tree, pear tree, jujube tree, peach tree, kiwi fruit tree, grape tree, citrus fruit tree, persimmon tree, plum tree, apricot tree and banana tree; woody plants such as pine, palm oil, and eucalyptus; flowering crops such as rose, gladiolus, gerbera, carnation, chrysanthemum, lily and tulip; and fodder crops such as ryegrass, red clover, orchardgrass, alfalfa, tall fescue and perennial ryegrass, but not be limited thereto. As a specific embodiment, the plant may be at least one selected from the group consisting of dicotyledonous plants such as arabidopsis, potato, eggplant, tobacco, red pepper, tomato, burdock, crown daisy, lettuce, balloon flower, spinach, chard, sweet potato, celery, carrot, water dropwort, parsley, Chinese cabbage, cabbage, radish, watermelon, oriental melon, cucumber, pumpkin, gourd, strawberry, soybean, mung bean, kidney bean, and pea; and monocotyledonous plants such as rice, wheat, barley, corn, sorghum, and the like, but not be limited thereto.

The algae, to which the present invention is applied, are not particularly limited to, but may be at least one prokaryotic algae or/or eukaryotic algae. For example, the algae may be at least one selected from the group consisting of cyanobacteria, green algae, red algae, brown algae, macroalgae, microalgae, and the like.

The cyanobacteria may include phylums Chroococcales (e.g., Aphanocapsa, Aphanothece, Chamaesiphon, Chondrocystis, Chroococcus, Chroogloeocystis, Crocosphaera, Cyanobacterium, Cyanobium, Cyanodictyon, Cyanosarcina, Cyanothece, Dactylococcopsis, Gloeocapsa, Gloeothece, Halothece, Johannesbaptistia, Merismopedia, Microcystis, Radiocystis, Rhabdoderma, Snowella, Synechococcus, Synechocystis, Thermosynechococcus, Woronichinia), Gloeobacteria, Nostocales (e.g., Microchaetaceae, Nostocaceae, Rivulariaceae, Scytonemataceae), Oscillatoriales (e.g., Arthronema, Arthrospira, Blennothrix, Crinalium, Geitlerinema, Halomicronema, Halospirulina, Hydrocoleum, Jaaginema, Katagnymene, Komvophoron, Leptolyngbya, Limnothrix, Lyngbya, Microcoleus, Oscillatoria, Phormidium, Planktothricoides, Planktothrix, Plectonema, Pseudanabaena, Pseudophormidium, Schizothrix, Spirulina, Starria, Symploca, Trichodesmium, Tychonema), Pleurocapsales (e.g., Chroococcidiopsis, Dermocarpa, Dermocarpella, Myxosarcina, Pleurocapsa, Solentia, Stanieria, Xenococcus), Prochlorales Stigonematales (e.g., Capsosira, Chlorogloeopsis, Fischerella, Hapalosiphon, Mastigocladopsis, Mastigocladus, Nostochopsis, Stigonema, Symphyonema, Symphonemopsis, Umezakia, Westiellopsis), and the like.

As another example of algae, Chlorophyta, Chlamydomonas, Volvacales, Dunaliella, Scenedesmus, Chlorella, or Hematococcm may be exemplified.

As other example of algae, Phaeodactylum tricornutum, Amphiprora hyaline, Amphora spp., Chaetoceros muelleri, Navicula saprophila, Nitzschia communis, Scenedesmus dimorphus, Scenedesmus obliquus, Tetraselmis suecica, Chlamydomonas reinhardtii, Chlorella vulgaris, Haematococcus pluvialis, Neochloris oleoabundans, Synechococcus elongatus, Botryococcus braunii, Gloeobacter violaceus, Synechocystis, Thermosynechococcus elongatus, Nannochloropsis oculata, Nannochloropsis salina, Nannochloropsis gaditana, Isochrysis galbana, Botryococcus sudeticus, Euglena gracilis, Neochloris oleoabundans, Nitzschia palea, Pleurochrysis carterae, Tetraselmis chuii, Pavlova spp., Aphanocapsa spp., Synechosystis spp., Nannochloris spp., and the like may be exemplified. However, it is not limited to kinds listed above, and algae belonging to other various genus and family may be comprised.

In an embodiment, the plant or algae with the herbicide-tolerant PPO or its variant provided herein may exhibit tolerance against two or more of PPO-inhibiting herbicides.

Therefore, the technology provided by this disclosure may be used to control weeds or remove undesired aquatic organisms by using at least two PPO-inhibiting herbicides sequentially or simultaneously.

One embodiment provides a method of controlling weeds in a cropland, comprising

providing the cropland with a plant comprising the herbicide-tolerant PPO protein, its variant, or a gene encoding the same as described above, and

applying an effective dosage of protoporphyrinogen IX oxidase-inhibiting herbicide to the cropland and/or the plant.

Another embodiment provides a method of removing an undesired aquatic organism from a culture medium, comprising:

providing a culture medium with algae comprising the herbicide-tolerant PPO protein, its variant, or a gene encoding the same described above, and

applying an effective dosage of protoporphyrinogen IX oxidase-inhibiting herbicide to the culture medium.

In addition, the herbicide-tolerant PPO protein, its variant, or a gene encoding the same provided herein may be used in combination of a second herbicide-tolerant polypeptide or a gene encoding the same.

Therefore, the plant or algae introduced with the herbicide-tolerant PPO provided herein may exhibit tolerance against two or more of herbicides which are different from each other in mechanism of action. In the present invention, two or more of different herbicides including the PPO-inhibiting herbicide, which are different from each other in mechanism of action, may be used sequentially or simultaneously, thereby controlling weeds and/or removing undesired aquatic organisms. Hereinafter, the herbicide which is different from the PPO-inhibiting herbicide in the mechanism of action is called “second herbicide”.

One embodiment provides a composition for conferring or enhancing herbicide tolerance of plants or algae, comprising the above-described herbicide-tolerant PPO protein, its variant, or a gene encoding the same; and a second herbicide-tolerant polypeptide or a gene encoding the same.

Another embodiment provides a transformant of plants or algae having herbicide tolerance, or a clone or progeny thereof, comprising the above-described herbicide-tolerant PPO protein, its variant, or a gene encoding the same; and a second herbicide-tolerant polypeptide or a gene encoding the same.

Another embodiment provides a method of preparing plants or algae having herbicide tolerance, comprising a step of introducing the above-described herbicide-tolerant PPO protein, its variant, or a gene encoding the same and a second herbicide-tolerant polypeptide or a gene encoding the same, into an alga, or a cell, protoplast, callus, hypocotyl, seed, cotyledon, shoot, or whole body of a plant.

Another embodiment provides a method of controlling weeds in a cropland, comprising

providing the cropland with a plant comprising the above-described herbicide-tolerant PPO protein, its variant, or a gene encoding the same, and a second herbicide-tolerant polypeptide or a gene encoding the same, and

applying effective dosages of protoporphyrinogen IX oxidase-inhibiting herbicide and the second herbicide to the cropland simultaneously or sequently in any order.

Another embodiment provides a method of removing an undesired aquatic organism from a culture medium, comprising

providing a culture medium with algae comprising the herbicide-tolerant PPO protein, its variant, or a gene encoding the same and a second herbicide-tolerant polypeptide or a gene encoding the same, and

applying effective dosages of protoporphyrinogen IX oxidase-inhibiting herbicide and the second herbicide to the culture medium simultaneously or sequently in any order.

For example, the plant or algae may further comprise the second herbicide-tolerance polypeptide or a gene encoding the same, thereby having acquired and/or enhanced tolerance against the second herbicide.

For example, the plant or alga further includes the second herbicide-tolerance polypeptide or a gene encoding thereof, thereby having novel and/or enhanced tolerance against the second herbicide.

For example, the second herbicide may include cell division-inhibiting herbicides, photosynthesis-inhibiting herbicides, amino acid synthesis-inhibiting herbicides, plastid-inhibiting herbicides, cell membrane-inhibiting herbicides, and/or any combinations thereof, but is not limited thereto. The second herbicide may be exemplified by glyphosate, glufosinate, dicamba, 2,4-D (2,4-dichlorophenoxyacetic acid), ALS (acetolactate synthase)-inhibiting herbicides (for example, imidazolidinone, sulfonylurea, triazole pyrimidine, sulphonanilide, pyrimidine thiobenzoate, etc.), photosystem II-inhibiting herbicides, phenylurea-based herbicides, plastid-inhibiting herbicides, bromoxynil-based herbicides, and/or any combinations thereof, but is not limited thereto.

For example, the second herbicide-tolerant polypeptide may be exemplified as one or more kinds selected from the group consisting of glyphosate herbicide-tolerant EPSPS (glyphosate tolerant 5-enolpyruvylshikimate-3-phosphate synthase), GOX (glyphosate oxidase), GAT (glyphosate-N-acetyltransferase) or glyphosate decarboxylase; glufosinate herbicide-tolerant PAT (phosphinothricin-N-acetyltransferase); dicamba herbicide-tolerant DMO (dicamba monooxygenase); 2,4-D herbicide-tolerant 2,4-D monooxygenase or AAD (aryloxyalkanoate dioxygenase); ALS-inhibiting sulfonylurea-based herbicide-tolerant ALS (acetolactate synthase), AHAS (acetohydroxyacid synthase), or AtAHASL (Arabidopsis thaliana acetohydroxyacid synthase large subunit); photosystem II-inhibiting herbicide-tolerant photosystem II protein D1; phenylurea-based herbicide-tolerant cytochrome P450; plastid-inhibiting herbicide-tolerant HPPD (hydroxylphenylpyruvate dioxygenase); bromoxynil herbicide-tolerant nitrilase; and any combinations thereof, but is not limited thereto.

Further, the gene encoding the second herbicide-tolerant polypeptide may be exemplified as one or more kinds selected from the group consisting of glyphosate herbicide-tolerant cp4 epsps, epsps (AG), mepsps, 2mepsps, goxv247, gat4601 or gat4621 gene; glufosinate herbicide-tolerant bar, pat or pat (SYN) gene; dicamba herbicide-tolerant dmo gene; 2,4-D herbicide-tolerant AAD-1 or AAD-12 gene; ALS-inhibiting sulfonylurea-based herbicide-tolerant ALS, GM-HRA, S4-HRA, ZM-HRA, Csr1, Csr1-1, Csr1-2, SurA or SurB; photosystem II-inhibiting herbicide-tolerant psba gene; phenylurea herbicide-tolerant CYP76B1 gene; isoxaflutole herbicide-tolerant HPPDPF W336 gene; bromoxynil herbicide-tolerant bxn gene; and any combinations thereof, but is not limited thereto.

### Advantageous Effects

A variant of herbicide-tolerant PPO protein or a gene encoding the same provided herein may be applied to a plant or algae, thereby conferring excellent herbicide tolerance traits to the plant or algae and/or enhancing the herbicide tolerance traits of the plant or algae. In addition, a selective control can be performed using herbicides, thereby economically controlling weeds or removing aquatic organisms.

### DESCRIPTION OF DRAWINGS

FIG. 1 is a map of pET303-CT-His vector.

FIG. 2 is a photograph showing cell growth level of PPO-deficient BT3 E. coli (BT3(ΔPPO)) transformant transformed with ApPPO1 wild type gene (indicated by ApPPO1WT), or various ApPPO1 mutant genes leading to a mutation of one amino acid, when treated with tiafenacil at a concentration of 0 μM (control), 50 μM, and 100 μM, respectively (upper), and saflufenacil at a concentration of 0 μM (control), 50 μM, and 100 μM, respectively (lower).

FIG. 3 is a photograph showing cell growth level of BT3(ΔPPO) transformant transformed with ApPPO1WT, or various ApPPO1 mutant genes leading to a mutation of one amino acid, when treated with flumioxazin at a concentration of 0 μM (control), 50 μM, and 200 μM, respectively (upper), and sulfentrazone at a concentration of 0 μM (control), 5 μM, and 25 μM, respectively (lower).

FIG. 4 is a photograph showing cell growth level of BT3(ΔPPO) transformant transformed with ApPPO1WT, or various ApPPO1 mutant genes leading to a mutation of one amino acid, when treated with fomesafen at a concentration of 0 μM (control), 5 μM, and 25 μM, respectively (upper), and acifluorfen at a concentration of 0 μM (control), 5 μM, and 25 μM, respectively (lower).

FIG. 5 is a photograph showing cell growth level of BT3(ΔPPO) transformant transformed with ApPPO1WT, or various ApPPO1 mutant genes leading to a mutation of one amino acid, when treated with pyraclonil at a concentration of 0 μM (control), 5 μM, and 25 μM, respectively (upper), and pentoxazone at a concentration of 0 μM (control), 5 μM, and 10 μM, respectively (lower).

FIG. 6 is a photograph showing cell growth level of BT3(ΔPPO) transformant transformed with ApPPO1WT, or various ApPPO1 mutant genes leading to a mutation of one amino acid, when treated with pyraflufen-ethyl at a concentration of 0 μM (control), 5 μM, and 10 μM, respectively.

FIGS. 7 to 12 are photographs showing cell growth level of BT3(ΔPPO) transformants transformed with ApPPO1 wild type gene (indicated by ApPPO1WT), or various ApPPO1 mutant genes leading to mutations of two or more amino acids as shown in Table 8, when treated with tiafenacil at a concentration of 0 μM (control), 50 μM, and 200 μM, respectively, flumioxazin at a concentration of 0 μM (control), 50 μM, and 100 μM, respectively, and sulfentrazone at a concentration of 0 μM (control), 200 μM, and 400 μM, respectively.

FIG. 13 is a photograph showing cell growth level of PPO-deficient BT3 E. coli (BT3(ΔPPO)) transformant transformed with MxPPO wild type gene (indicated by MxPPOWT), or various MxPPO mutant genes leading to a mutation of one amino acid, when treated with tiafenacil at a concentration of 0 μM (control), 200 μM, and 2000 μM, saflufenacil at a concentration of 0 μM (control), 100 μM, and 200 μM, and flumioxazin at a concentration of 0 μM (control), 50 μM, and 100 μM, respectively.

FIG. 14 is a photograph showing cell growth level of BT3(ΔPPO) transformant transformed with MxPPOWT, or various MxPPO mutant genes leading to mutations of two or more amino acids as shown in Table 10, when treated with tiafenacil at a concentration of 0 μM (control) and 2000 μM, respectively.

FIGS. 15 to 17 are a photograph showing cell growth level of BT3(ΔPPO) transformant transformed with MxPPOWT, or various MxPPO mutant genes leading to mutations of two or more amino acids as shown in Table 10, when treated with flumioxazin at a concentration of 0 μM (control). 200 μM, and 400 μM, respectively.

FIGS. 18 to 20 are a photograph showing cell growth level of BT3(ΔPPO) transformant transformed with MxPPOWT, or various MxPPO mutant genes leading to mutations of two or more amino acids as shown in Table 10, when treated with sulfentrazone at a concentration of 0 μM (control), 200 μM, and 1000 μM, respectively.

FIGS. 21 and 22 are a photograph showing cell growth level of BT3(ΔPPO) transformant transformed with MxPPOWT, or various MxPPO mutant genes made by multiple amino acid changes as shown in Table 10, when treated with flumioxazin at a concentration of 0 μM (control), 400 μM, and 1000 μM, respectively.

FIGS. 23 and 24 are a photograph showing cell growth level of BT3(ΔPPO) transformant transformed with MxPPOWT, or various MxPPO mutant genes made by multiple amino acid changes as shown in Table 10, when treated with sulfentrazone at a concentration of 0 μM (control), 2000 μM, and 4000 μM, respectively.

FIG. 25 is a map of pMAL-c2X vector.

FIG. 26 is a photograph showing seed germination results observed at the 6^{th} day after sowing the seeds of A. thaliana wild type (Col-0) or transformants of ApPPO1 nutant genes in herbicide-containing medium.

FIG. 27 is a photograph showing seed germination results of observed at the 6^{th} day after sowing the seeds of A. thaliana wild type (Col-0) or transformants of an MxPPO and an MxPPO mutant gene in herbicide-containing medium.

### MODE FOR INVENTION

Hereinafter, the present invention will be described in detail with reference to Examples. However, these Examples are for illustrative purposes only, and the invention is not intended to be limited by these Examples.

### Example 1. Verification of Herbicide Tolerance of ApPPO1 and MxPPO Isolated from Prokaryotes

PPO gene sequences were obtained from Genebank database of two strains, Auxenochlorella protothecoides and Myxococcus xanthus, respectively. For encoding the PPO protein (ApPPO1; SEQ ID NO: 1) from Auxenochlorella protothecoides, the PPO gene designated as ApPPO1 was isolated from Auxenochlorella protothecoides, and optimized to have the nucleic acid sequence of SEQ ID NO: 7. For encoding the PPO protein (MxPPO; SEQ ID NO: 3) Myxococcus xanthus designated as MxPPO was isolated from Myxococcus xanthus and optimized to have the nucleic acid sequence of SEQ ID NO: 8. In order to obtain the herbicide-binding structure of PPO protein, the herbicides including tiafenacil, saflufenacil, flumioxazin, and sulfentrazone and the PPO proteins including ApPPO1 and MxPPO were used. Homology model of ApPPO1 was constructed from CyPPO10 (the PPO protein originated from Thermosynechococcus elongatus BP-1; SEQ ID NO: 5) structure using SWISS-MODEL protein structure modelling server (https://swissmodel.expasy.org/). The structure information of MxPPO was used from RCSB protein data bank (https://www.rcsb.org/pdb/home/home.do) (PDB ID: 2IVE)Herbicide-interacting structural information of ApPPO1 and MxPPO were superimposed with CyPPO10 bound with herbicides (tiafenacil, saflufenacil, flumioxazin, and sulfentrazone).

Herbicide-binding information of CyPPO10 was obtained by following procedures: CyPPO10 protein (SEQ ID NO: 5) and tiafenacil, saflufenacil, flumioxazin, and sulfentrazone were examined as the representative protein and herbicides, respectively. The gene encoding the CyPPO10 protein (SEQ ID NO: 6) was cloned to pET29b vector (Catalog Number: 69872-3; EMD Biosciences), and CyPPO10 protein was expressed in E. coli. The expressed CyPPO10 protein was purified through nickel affinity chromatography, to which tiafenacil, saflufenacil, flumioxazin or sulfentrazone was added respectively and herbicide-bound PPO crystals were obtained. Then, the crystals were used for X-ray diffraction by synchrotron radiation accelerator. X-ray diffraction data of the 2.4A resolution of CyPPO10-herbicide complex crystals was obtained, and the three-dimensional structure was determined. Binding information was obtained through analyzing the amino acid residues of CyPPO10 interacting with herbicides.

Using the information of herbicide-interacting amino acids derived from the structure of CyPPO10-herbicide complexes, information of ApPPO1 and MxPPO amino acid residues which possibly lower the binding affinity of herbicides through mutations were determined.

As results, amino acid residues including R140, F209, V213, A215, G216, V360, S362, F386, L389, L399, I402 and Y422 of ApPPO1 protein (SEQ ID NO: 1) were involved to interact with herbicides (tiafenacil, saflufenacil, flumioxazin, and sulfentrazone) and those including R95, V164, I168, A170, G171, I311, V313, F329, L332, L342, I345 and M365 of MxPPO protein (SEQ ID NO: 3) were involved to interact with herbicides (tiafenacil, saflufenacil, flumioxazin, and sulfentrazone).

### Example 2. Construction of PPO Variants

In order to enhance PPO-inhibiting herbicide tolerance of ApPPO1 and MxPPO, a mutation(s) at the position interacting with herbicide obtained in the Example 1 was introduced, respectively. Each PPO gene was codon-optimized and synthesized (Cosmogenetech Co., Ltd.) for efficient herbicide tolerance test using BT3, a PPO-deficient E. coli stain.

Detailed experimental procedure was as follows:

Using primers listed in Table 2, PCR was carried out to amplify PPO genes under following condition.

PCR reaction mixture

Template (synthetic DNA of ApPPO1 and MxPPO) 1 μl

10× buffer 5 μl

dNTP mixture (10 mM each) 1 μl

Forward primer (10 μM) 1 μl

Reverse primer (10 μM) 1 μl

DDW 40 μl

TABLE 2
Primer list for cloning of ApPPO1
and MxPPO in pET303-CT His

| | | | | SED |
|---|---|---|---|---|
| | | | Se- | ID |
| Gene | Strain | Primer | quence | No. |
| ApPPO | Auxenochlorella | ApPPO1_ | CCCCTCTA | 9 |
| 1 | protothecoides | XbaIF | GAATGGCC | |
| | | | GAGTACGA | |
| | | | CGTTGT | |
| | | | TAACGT | |
| | | ApPPO1_ | CCCCCTCG | 10 |
| | | XhoIR | AGGGTTGC | |
| | | | CAGACTTT | |
| | | | TAACGT | |
| MxPPO | Myxococcus | MxPPO1_ | CCCCTCTA | 11 |
| | xanthus | XbaIF | GAATGCAC | |
| | | | CATATGCC | |
| | | | CCGAAC | |
| | | | TAACGT | |
| | | MxPPO1_ | CCCCCTCG | 12 |
| | | XhoIR | AGAGGCGC | |
| | | | GTGTGATG | |
| | | | TATTAC | |

Pfu-X (Solgent, 2.5 units/μl) 1 μl

Total 50 μl

TABLE 1
PCR reaction condition

| | | | | |
|---|---|---|---|---|
| 94° C. | 4 | min. | 1 | cycle |
| 94° C. | 30 | sec. | 25 | cycles |
| 56° C. | 30 | sec. | | |
| 72° C. | 1.5 | min. | | |
| 72° C. | 5 | min. | 1 | cycle |
| 4° C. | 5 | min. | 1 | cycle |

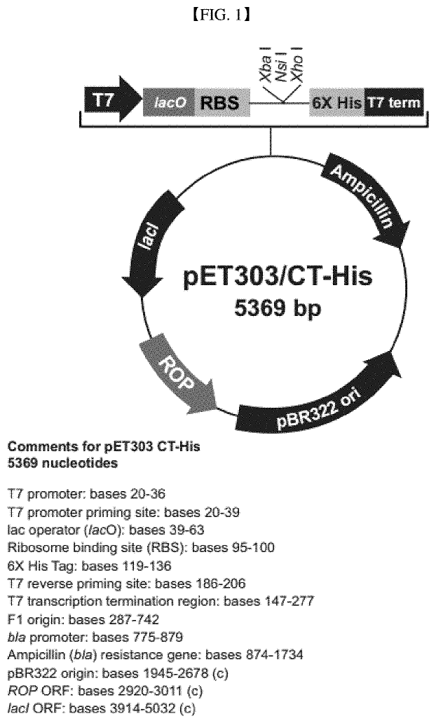

Amplified PCR products above and pET303-CT His vector (VT0163; Novagen; FIG. 1) were digested with XbaI and XhoI restriction enzymes, and ligated to construct pET303-ApPPO1 and pET303-MxPPO plasmids using T4 DNA ligase(RBC, 3 units/μl).

ApPPO1 and MxPPO genes cloned in pET303-CT His vector were mutated through site-directed mutagenesis using primers listed in Tables 4 and 5, respectively.

PCR reaction mixture

Template 1 μl

10× buffer 5 μl

dNTP mixture (10 mM each) 1 μl

Forward primer (10 μM) 1 μl

Reverse primer (10 μM) 1 μl

DDW 40 μl

Pfu-X (Solgent, 2.5 units/μl) 1 μl

Total 50 μl

TABLE 3
PCR reaction condition

| | | | | |
|---|---|---|---|---|
| 94° C. | 2 | min. | 1 | cycle |
| 94° C. | 30 | sec. | 17-25 | cycles |
| 65° C. | 40 | sec. | | |
| 72° C. | 3.5 | min. | | |
| 72° C. | 5 | min. | 1 | cycle |
| 4° C. | 5 | min. | 1 | cycle |

TABLE 4
Primer list for mutagenesis
of ApPPO1 gene

| | | | SEQ |
|---|---|---|---|
| ApPPO1 | Primer Sequence | | ID |
| mutation | (5′-> 3′) | | NO |
| Y422M | F | CTCT | 13 |
| | | TGTC | |
| | | ACTT | |
| | | TATG | |
| | | GGGG | |
| | | GGCT | |
| | | ACCA | |
| | | ACAC | |
| | R | CCCC | 14 |
| | | CATA | |
| | | AAGT | |
| | | GACA | |
| | | AGAG | |
| | | CAGC | |
| | | ACCT | |
| | | TTCC | |
| Y422L | F | CTCT | 15 |
| | | TGTC | |
| | | ACTT | |
| | | TTTG | |
| | | GGGG | |
| | | GGCT | |
| | | AGCA | |
| | | ACAC | |
| | R | CCCC | 16 |
| | | CAAA | |
| | | AAGT | |
| | | GACA | |
| | | AGAG | |
| | | CAGC | |
| | | ACCT | |
| | | TTCC | |
| Y422C | F | TCTT | 17 |
| | | GTCA | |
| | | TGTT | |
| | | TTGG | |
| | | GGGG | |
| | | GCTA | |
| | | CCAA | |
| | | CAC | |
| | R | CCCC | 18 |
| | | CAAA | |
| | | ACAT | |
| | | GACA | |
| | | AGAG | |
| | | CAGC | |
| | | ACCT | |
| | | TTCC | |
| Y422V | F | CTCT | 19 |
| | | TGTC | |
| | | AGTT | |
| | | TTTG | |
| | | GGGG | |
| | | GGCT | |
| | | ACCA | |
| | | ACAC | |
| | R | CCCC | 20 |
| | | CAAA | |
| | | AACT | |
| | | GACA | |
| | | AGAG | |
| | | CAGC | |
| | | ACCT | |
| | | TTC | |
| Y422I | F | CTCT | 21 |
| | | TGTC | |
| | | AATT | |
| | | TTTG | |
| | | GGGG | |
| | | GGCT | |
| | | ACCA | |
| | | ACAC | |
| | R | CCCC | 99 |
| | | AAAA | |
| | | ATTG | |
| | | ACAA | |
| | | GAGC | |
| | | AGCA | |
| | | CCTT | |
| | | TCC | |
| Y422T | F | GTGC | 23 |
| | | TGCT | |
| | | CTTG | |
| | | TCAA | |
| | | CCTT | |
| | | TGGG | |
| | | GGGG | |
| | | CTAC | |
| | | C | |
| | R | GGTA | 24 |
| | | GCCC | |
| | | CCCC | |
| | | AAAG | |
| | | GTTG | |
| | | ACAA | |
| | | GAGC | |
| | | AGC | |
| | | AC | |
| A215L | F | GGGT | 25 |
| | | TTAC | |
| | | CTCG | |
| | | GCGA | |
| | | CCCG | |
| | | GCTA | |
| | | AGTT | |
| | | GAG | |
| | R | GTCG | 26 |
| | | CCGA | |
| | | GGTA | |
| | | AACC | |
| | | CCGC | |
| | | TGCA | |
| | | AAAC | |
| | | GGC | |
| A215C | F | CiGG | 27 |
| | | TTTA | |
| | | CTGC | |
| | | GGCG | |
| | | ACCC | |
| | | GGCT | |
| | | AAGT | |
| | | TGAG | |
| | R | GTCG | 28 |
| | | CCGC | |
| | | AGTA | |
| | | AACC | |
| | | CCGC | |
| | | TGCA | |
| | | AAAC | |
| | | GGC | |
| V360M | F | CCCT | 29 |
| | | CCCA | |
| | | TGGC | |
| | | ATCT | |
| | | GTAG | |
| | | CATT | |
| | | ATCT | |
| | | TACC | |
| | R | TACA | 30 |
| | | GATG | |
| | | CCAT | |
| | | GGGA | |
| | | GGGT | |
| | | AATA | |
| | | GATA | |
| | | GAG | |
| | | C | |
| R140A | F | GATC | 31 |
| | | CGAA | |
| | | GGCG | |
| | | CCCG | |
| | | CGTA | |
| | | CGTT | |
| | | TATT | |
| | | GGGG | |
| | | TG | |
| | R | CACC | 32 |
| | | CCAA | |
| | | TAAA | |
| | | CGTA | |
| | | CGCG | |
| | | GGCG | |
| | | CCTT | |
| | | CGGA | |
| | | TC | |
| F209A | F | CGAC | 33 |
| | | TTAT | |
| | | AGAG | |
| | | CCGG | |
| | | CGTG | |
| | | CAGC | |
| | | GGGG | |
| | | TTTA | |
| | | C | |
| | R | GTAA | 34 |
| | | ACCC | |
| | | CGCT | |
| | | GCAC | |
| | | GCCG | |
| | | GCTC | |
| | | TATA | |
| | | AGTC | |
| | | G | |
| V213C | F | CCGT | 35 |
| | | TTTG | |
| | | CAGC | |
| | | GGGT | |
| | | GCTA | |
| | | CGCC | |
| | | GGCG | |
| | | ACCC | |
| | | G | |
| | R | CGGG | 36 |
| | | TCGC | |
| | | CGGC | |
| | | GTAG | |
| | | CACC | |
| | | CGCT | |
| | | GCAA | |
| | | AACG | |
| | | G | |
| F386V | F | GGTC | 37 |
| | | ACCT | |
| | | AGCG | |
| | | GGCG | |
| | | TGGG | |
| | | CCAG | |
| | | CTAC | |
| | | ACCC | |
| | | TC | |
| | R | GAGG | 38 |
| | | GTGT | |
| | | AGCT | |
| | | GGCC | |
| | | CACG | |
| | | CCCG | |
| | | CTAG | |
| | | GTGA | |
| | | CC | |
| L389T | F | GCGG | 39 |
| | | GCTT | |
| | | TGGC | |
| | | CAGA | |
| | | CCCA | |
| | | CCCT | |
| | | CGTA | |
| | | CTCA | |
| | | G | |
| | R | CTGA | 40 |
| | | GTAC | |
| | | GAGG | |
| | | GTGG | |
| | | GTCT | |
| | | GGCC | |
| | | AAAG | |
| | | CCCG | |
| | | C | |
| I402T | F | CACC | 41 |
| | | ACTC | |
| | | TGGG | |
| | | CACT | |
| | | ACCT | |
| | | ATGC | |
| | | CTCA | |
| | | AGCT | |
| | | TA | |
| | R | TAAG | 42 |
| | | CTTG | |
| | | AGGC | |
| | | ATAG | |
| | | GTAG | |
| | | TGCC | |
| | | CAGA | |
| | | GTGG | |
| | | TG | |
| V360I | F | CTAT | 43 |
| | | TACC | |
| | | CTCC | |
| | | CATC | |
| | | GCAT | |
| | | CTGT | |
| | | AGCA | |
| | | TTAT | |
| | | C | |
| | R | GATA | 44 |
| | | ATGC | |
| | | TACA | |
| | | GATG | |
| | | CGAT | |
| | | GGGA | |
| | | GGGT | |
| | | AATA | |
| | | G | |
| V360L | F | TACC | 45 |
| | | CTCC | |
| | | CCTC | |
| | | GCAT | |
| | | CTGT | |
| | | AGCA | |
| | | TTAT | |
| | | CTTA | |
| | | C | |
| | R | CAGA | 46 |
| | | TGCG | |
| | | AGGG | |
| | | GAGG | |
| | | GTAA | |
| | | TAGA | |
| | | TAGA | |
| | | GC | |
| S362V | F | TACC | 47 |
| | | CTCC | |
| | | CGTC | |
| | | GCAG | |
| | | TTGT | |
| | | AGCA | |
| | | TTAT | |
| | | CTTA | |
| | | C | |
| | R | GTAA | 48 |
| | | GATA | |
| | | ATGC | |
| | | TACA | |
| | | ACTG | |
| | | CGAC | |
| | | GGGA | |
| | | GGGT | |
| | | A | |
| V213C + | F | TTGC | 49 |
| A215C | | AGCG | |
| | | GGTG | |
| | | CTAC | |
| | | TGCG | |
| | | GCGA | |
| | | CCCG | |
| | | GCTA | |
| | | AGT | |
| | R | ACTT | 50 |
| | | AGCC | |
| | | GGGT | |
| | | CGCC | |
| | | GCAG | |
| | | TAGC | |
| | | ACCC | |
| | | GCTG | |
| | | CAA | |
| V213C + | F | TTGC | 51 |
| A215L | | AGCG | |
| | | GGTG | |
| | | CTAC | |
| | | CTTG | |
| | | GCGA | |
| | | CCCG | |
| | | GCTA | |
| | | AGT | |
| | R | ACTT | 52 |
| | | AGCC | |
| | | GGGT | |
| | | CGCC | |
| | | AAGG | |
| | | TAGC | |
| | | ACCC | |
| | | GCTG | |
| | | CAA | |

TABLE 5
Primer list for mutagenesis
of MxPPO gene

| | Primer | | SEQ |
|---|---|---|---|
| MxPPO | Sequence | | ID |
| mutation | (5′-> 3′) | | NO |
| M365T | F | ACTC | 53 |
| | | ATGT | |
| | | ACGG | |
| | | TGGG | |
| | | GGGT | |
| | | GCAA | |
| | | GACA | |
| | | ACC | |
| | R | CCCC | 54 |
| | | ACCG | |
| | | TACA | |
| | | TGAG | |
| | | TATA | |
| | | AGAC | |
| | | ACGC | |
| | | CCAC | |
| M365L | F | TACT | 55 |
| | | CATG | |
| | | TCTG | |
| | | GTGG | |
| | | GGGG | |
| | | TGCA | |
| | | AGAC | |
| | | AACC | |
| | R | CCCA | 56 |
| | | CCAG | |
| | | ACAT | |
| | | GAGT | |
| | | ATAA | |
| | | GACA | |
| | | CGCC | |
| | | CAC | |
| M365C | F | TACT | 57 |
| | | CATG | |
| | | TTGC | |
| | | GTGG | |
| | | GGGG | |
| | | TGCA | |
| | | AGAC | |
| | | AACC | |
| | R | CCCC | 58 |
| | | CACG | |
| | | CAAC | |
| | | ATGA | |
| | | GTAT | |
| | | AAGA | |
| | | CACG | |
| | | CCC | |
| M365V | F | TACT | 59 |
| | | CATG | |
| | | TGTG | |
| | | GTGG | |
| | | GGGG | |
| | | TGCA | |
| | | AGAC | |
| | | AACC | |
| | R | CCCC | 60 |
| | | CCAC | |
| | | CACA | |
| | | CATG | |
| | | AGTA | |
| | | TAAG | |
| | | ACAC | |
| | | GCCC | |
| M365I | F | GTCT | 61 |
| | | TATA | |
| | | CTCA | |
| | | TGTA | |
| | | TCGT | |
| | | GGGG | |
| | | GGTG | |
| | | CAAG | |
| | | AC | |
| | R | GTCT | 62 |
| | | TGCA | |
| | | CCCC | |
| | | CCAC | |
| | | GATA | |
| | | CATG | |
| | | AGTA | |
| | | TAAG | |
| | | AC | |
| R95A | F | GCAA | 63 |
| | | AGAG | |
| | | AGCT | |
| | | TATG | |
| | | TCTA | |
| | | CACG | |
| | | CGAG | |
| | | GACG | |
| | R | GTAG | 64 |
| | | ACAT | |
| | | AAGC | |
| | | TCTC | |
| | | TTTG | |
| | | CAGC | |
| | | CGGA | |
| | | TCGG | |
| | | C | |
| VI64A | F | TAGA | 65 |
| | | TGCA | |
| | | GCGC | |
| | | AGAC | |
| | | AGGG | |
| | | ATAT | |
| | | ATGC | |
| | | CGG | |
| | R | CTGT | 66 |
| | | CTGC | |
| | | GCTG | |
| | | CATC | |
| | | TAAT | |
| | | AGAA | |
| | | CTTG | |
| | | GG | |
| I168C | F | CAGA | 67 |
| | | CAGG | |
| | | GTGC | |
| | | TATG | |
| | | CCGG | |
| | | AGAT | |
| | | GTTG | |
| | | AGC | |
| | R | TCCG | 68 |
| | | GCAT | |
| | | AGCA | |
| | | CCCT | |
| | | GTCT | |
| | | GCAC | |
| | | TGCA | |
| | | TCTA | |
| | | A | |
| A170C | F | GGGA | 69 |
| | | TATA | |
| | | TTGC | |
| | | GGAG | |
| | | ATGT | |
| | | TGAG | |
| | | CAAT | |
| | | TATC | |
| | R | ACAT | 70 |
| | | CTCC | |
| | | GCAA | |
| | | TATA | |
| | | TCCC | |
| | | TGTC | |
| | | TGCA | |
| | | CTGC | |
| A170L | F | GGGA | 71 |
| | | TATA | |
| | | TCTC | |
| | | GGAG | |
| | | ATGT | |
| | | TGAG | |
| | | CAAT | |
| | | TATC | |
| | R | ACAT | 72 |
| | | CTCC | |
| | | GAGA | |
| | | TATA | |
| | | TCCC | |
| | | TGTC | |
| | | TGCA | |
| | | CTGC | |
| I311M | F | TGCC | 73 |
| | | CCCA | |
| | | TGGC | |
| | | TGTA | |
| | | GTTC | |
| | | ATCT | |
| | | CGGA | |
| | | TTC | |
| | R | AACT | 74 |
| | | ACAG | |
| | | CCAT | |
| | | GGGG | |
| | | GCAT | |
| | | AGGC | |
| | | GATA | |
| | | CC | |
| F329V | F | CGAT | 75 |
| | | GGGG | |
| | | TCGG | |
| | | TTTT | |
| | | TTAG | |
| | | TGCC | |
| | | GGCG | |
| | | GAGG | |
| | R | AAAA | 76 |
| | | AACC | |
| | | GACC | |
| | | CCAT | |
| | | CGGG | |
| | | CGCC | |
| | | GGTA | |
| | | AAG | |
| L332T | F | TTCG | 77 |
| | | GTTT | |
| | | TACA | |
| | | GTGC | |
| | | CGGC | |
| | | GGAG | |
| | | GAAC | |
| | | AG | |
| | R | CCGG | 78 |
| | | CACT | |
| | | GTAA | |
| | | AACC | |
| | | GAAC | |
| | | CCAT | |
| | | CGGG | |
| | | CGC | |
| I345T | F | GGGT | 79 |
| | | GCCA | |
| | | CTCA | |
| | | TGCT | |
| | | TCCA | |
| | | CGAC | |
| | | TTTC | |
| | | CCG | |
| | R | GAAG | 80 |
| | | CATG | |
| | | AGTG | |
| | | GCAC | |
| | | CCAA | |
| | | CATC | |
| | | CTTC | |
| | | GCTG | |
| I168C + | F | GTGC | 81 |
| A170C | | AGAC | |
| | | AGGG | |
| | | TGCT | |
| | | ATTG | |
| | | CGGA | |
| | | GATG | |
| | | TTGA | |
| | | G | |
| | R | CTCA | 82 |
| | | ACAT | |
| | | CTCC | |
| | | GCAA | |
| | | TAGC | |
| | | ACCC | |
| | | TGTC | |
| | | TGCA | |
| | | C | |

One μl of DpnI (NEB) was treated to each 10 μl of PCR products, and incubated at 37° C. for 30 minutes. DH5alpha competent cell (Biofact Co., Ltd.) was transformed with reaction solution through heat shock method, and was cultured in LB agar media containing carbenicillin (Gold Biotechnology Co., Ltd.). After plasmids were prepared from transformed E. coli, they were sequenced (Cosmogenetech, Co., Ltd.) and confirmed to have correct mutations.

### Example 3. Verification of PPO-Inhibiting Herbicide Tolerance of PPO Variants (Test in E. coli)

The mutated CyPPO gene obtained from the Example 2 was transformed to BT3 (ΔPPO) strain which is deficient of PPO activity and cultured in LB media with PPO-inhibiting herbicide, thereby examining whether growth of transformed BT3 was not inhibited.

BT3 (ΔPPO) strain was provided by Hokkaido University (Japan) and it is an E. coli strain which is deficient in hemG-type PPO and has kanamycin resistance (refer to “Watanabe N, Che FS, Iwano M, Takayama S, Yoshida S, Isogai A. Dual targeting of spinach protoporphyrinogen IX oxidase II to mitochondria and chloroplasts by alternative use of two in-frame initiation codons, J. Biol. Chem. 276(23):20474-20481, 2001; Che FS, Watanabe N, Iwano M, Inokuchi H, Takayama S, Yoshida S, Isogai A. Molecular Characterization and Subcellular Localization of Protoporphyrinogen IX oxidase in Spinach Chloroplasts, Plant Physiol. 124(1):59-70, 2000”).

Detailed experimental procedure was as follows:

BT3 competent cells were transformed with the pET303-ApPPO1 and pET303-MxPPO plasmids and those with a mutation(s) constructed in Example 2 respectively, and were cultured in LB agar media containing carbenicillin (Gold Biotechnology, Co., Ltd.).

Single colony of E. coli transformed with each CyPPO gene was cultured in 3 ml of LB broth containing carbenicillin overnight, and then was subcultured until absorbance (OD₆₀₀) reached 0.5 to 1. Then, it was diluted with LB broth to OD₆₀₀=0.5. Again, the diluted solution was serially diluted 4 times by a factor of one tenth.

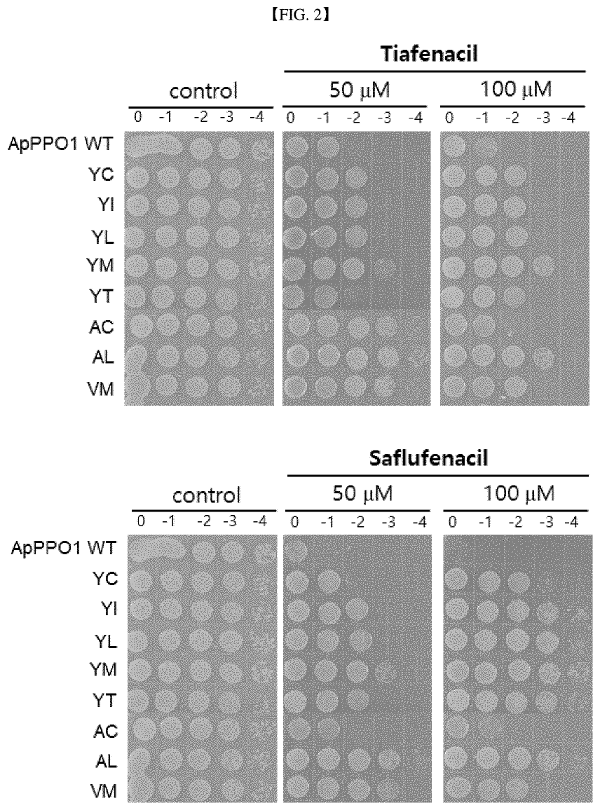

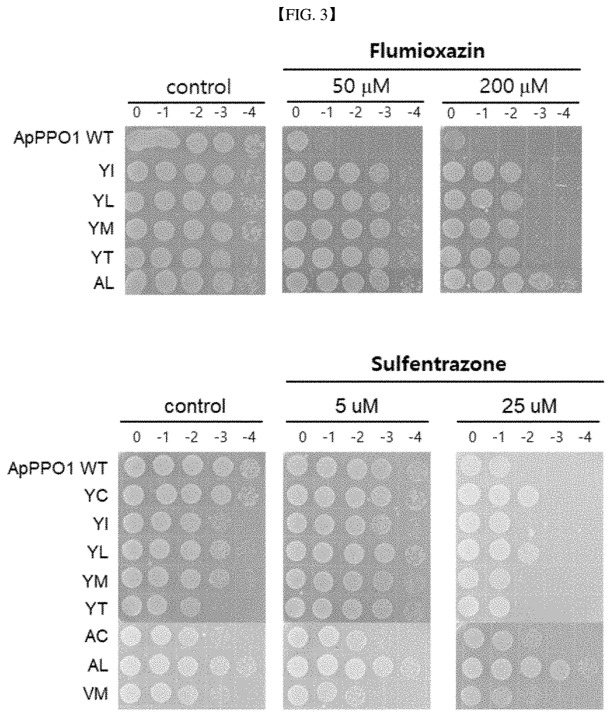

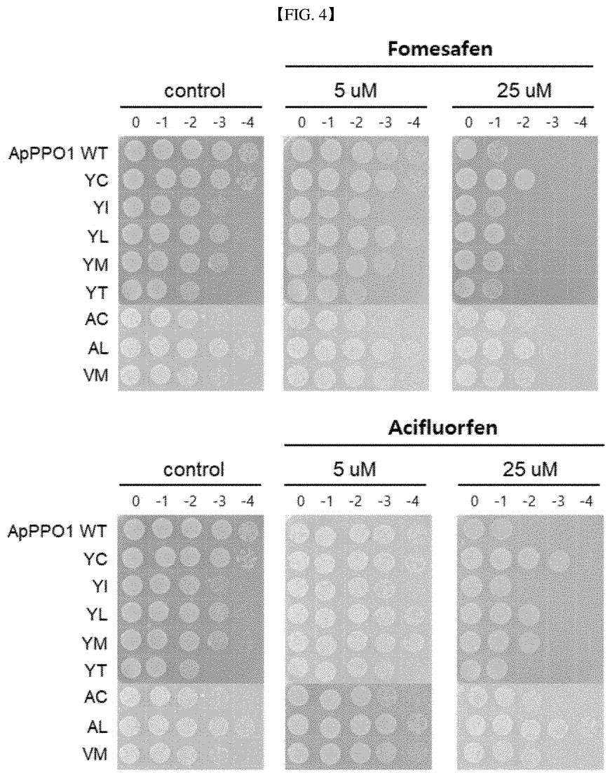

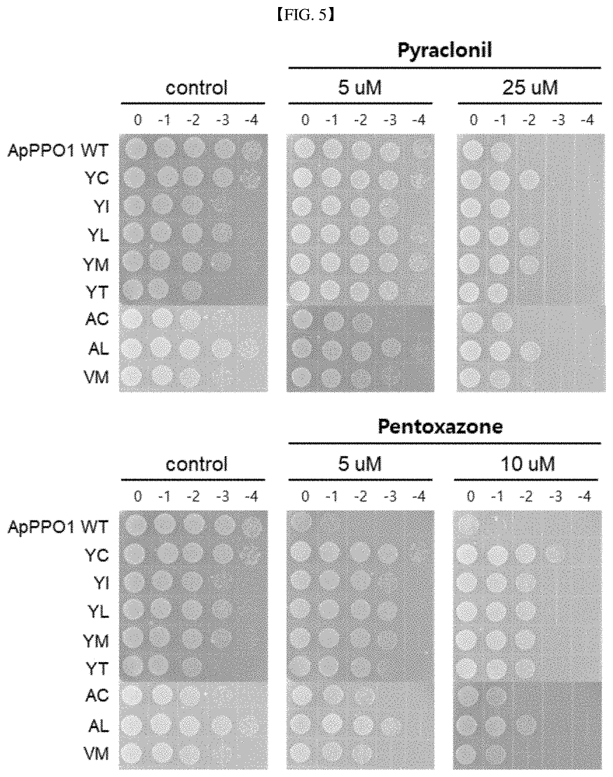

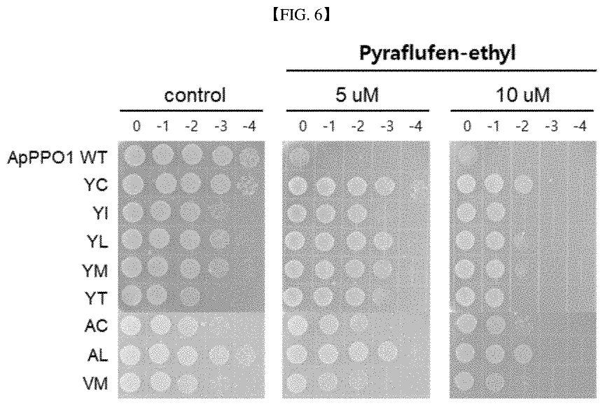

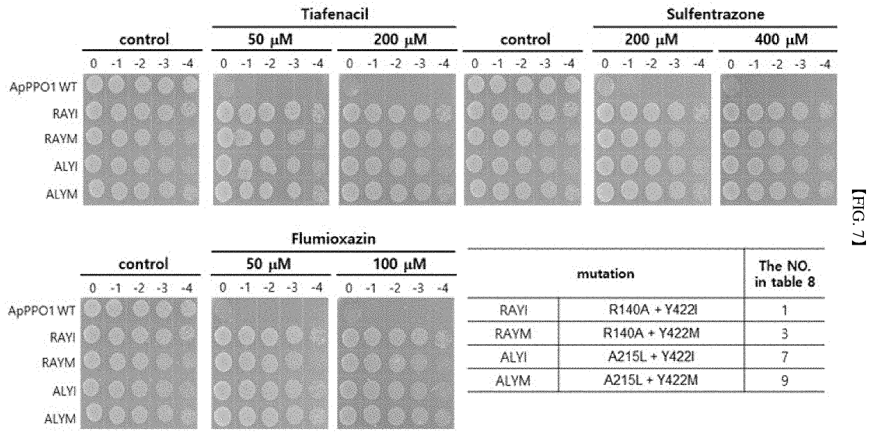

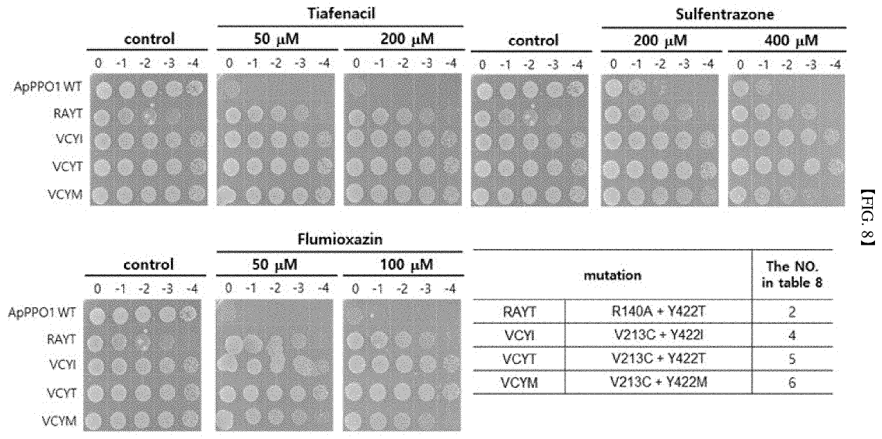

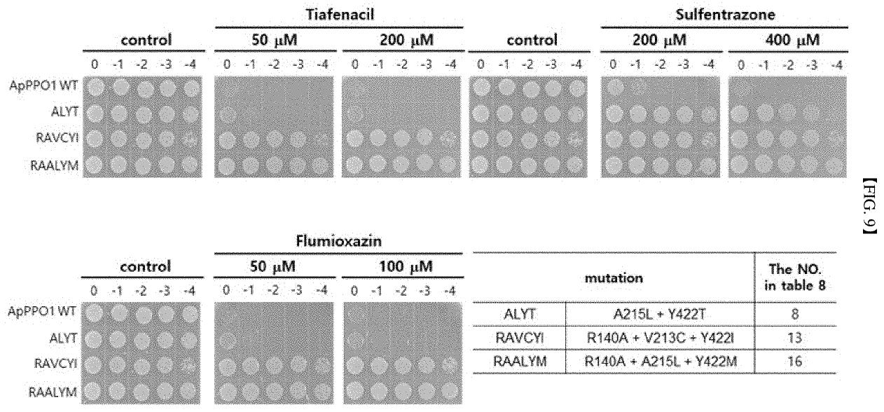

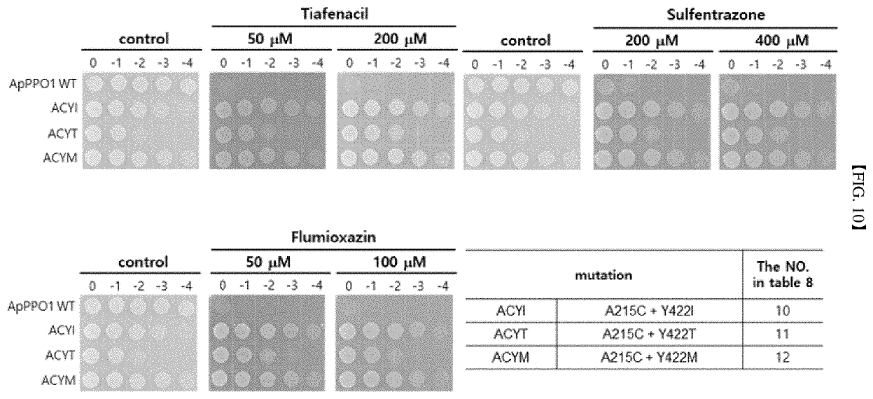

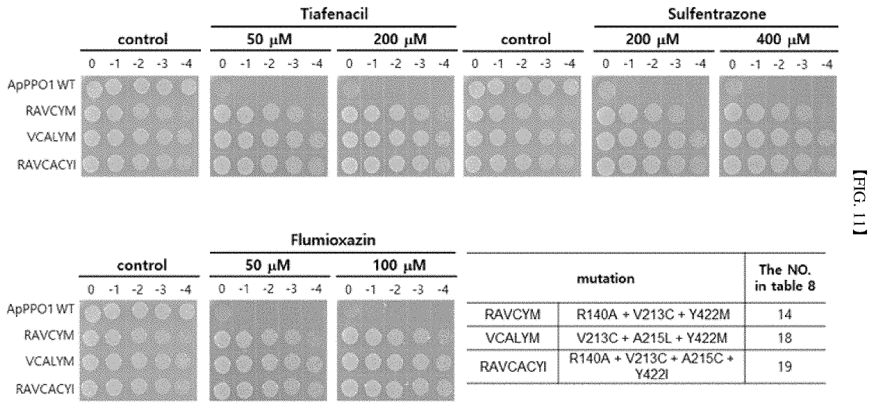

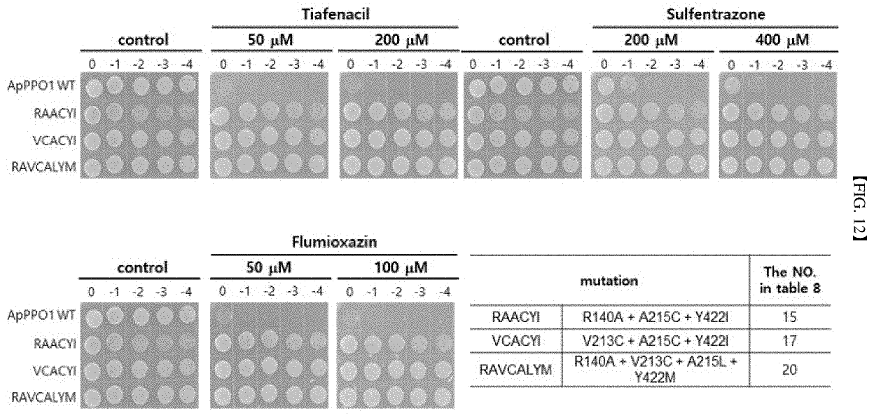

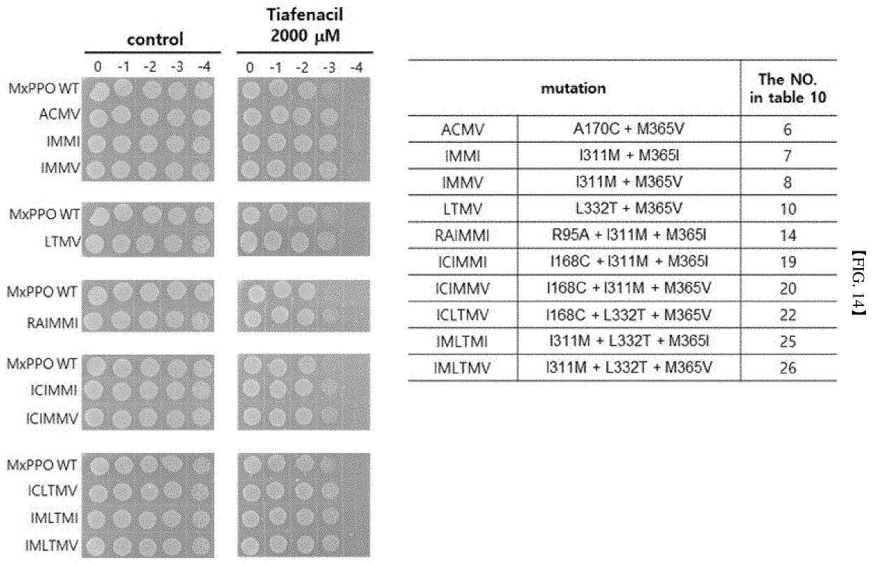

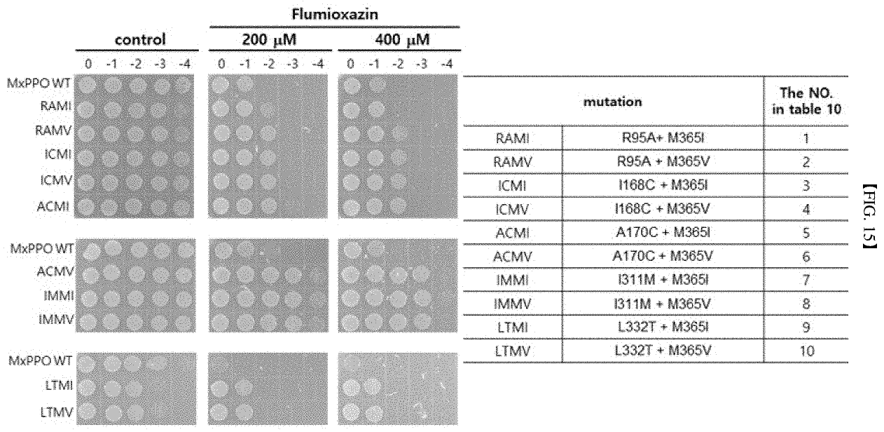

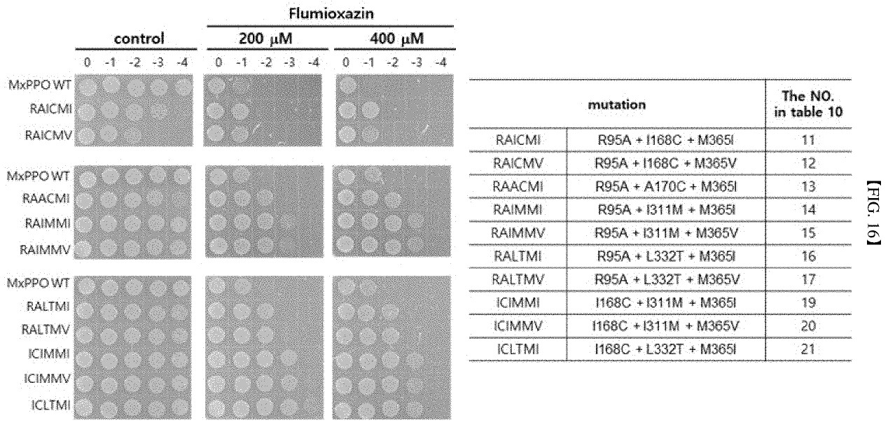

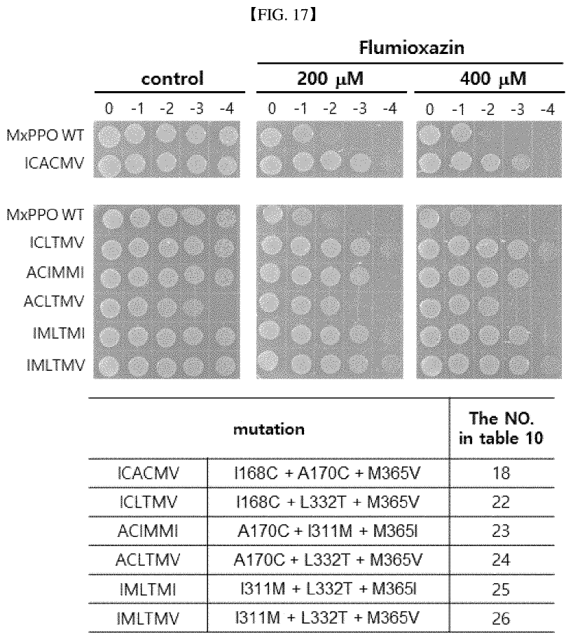

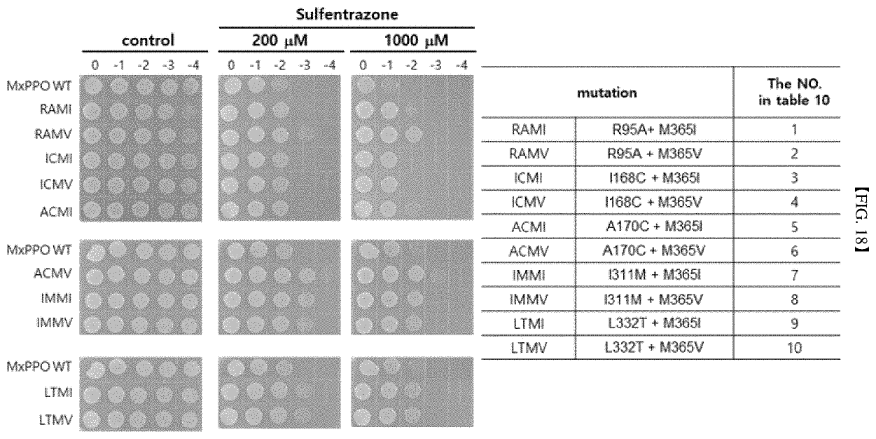

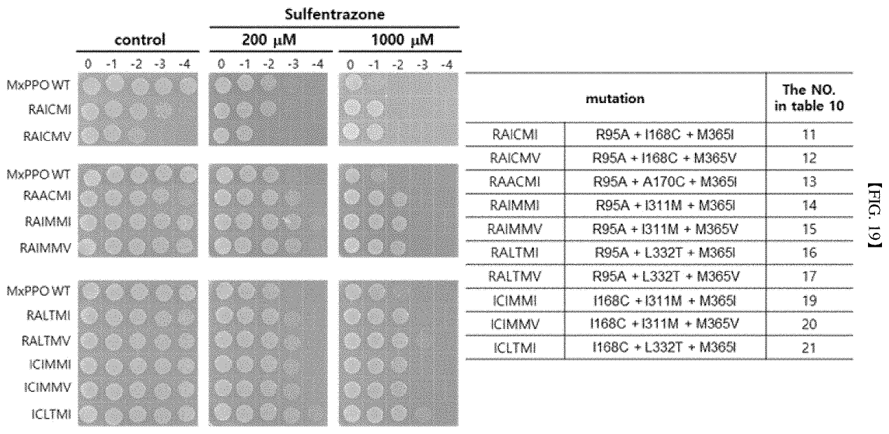

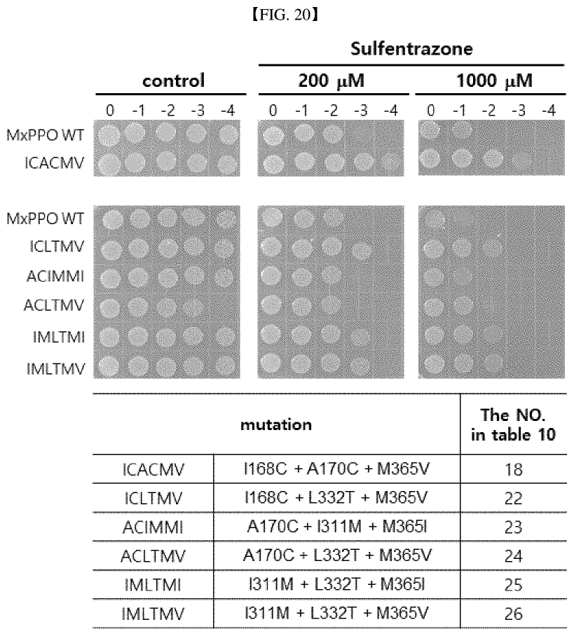

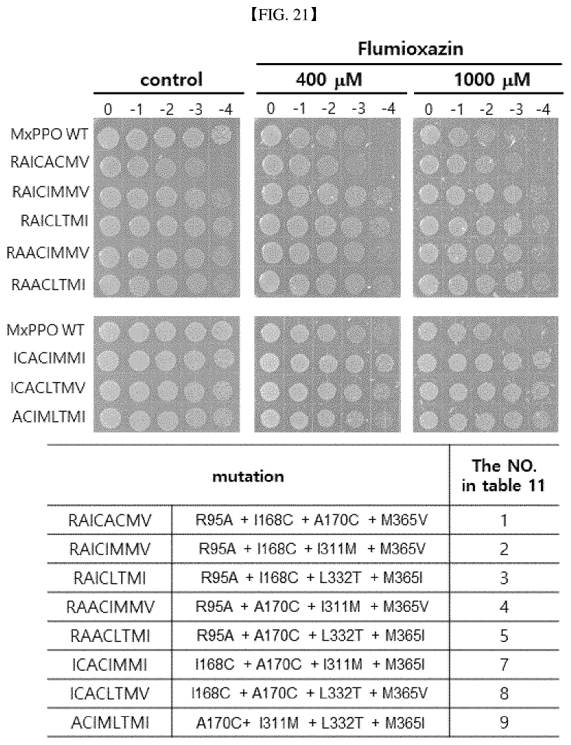

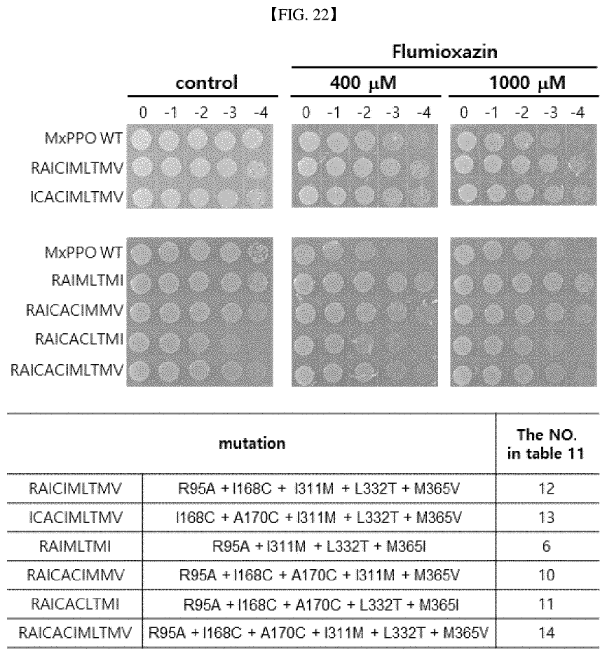

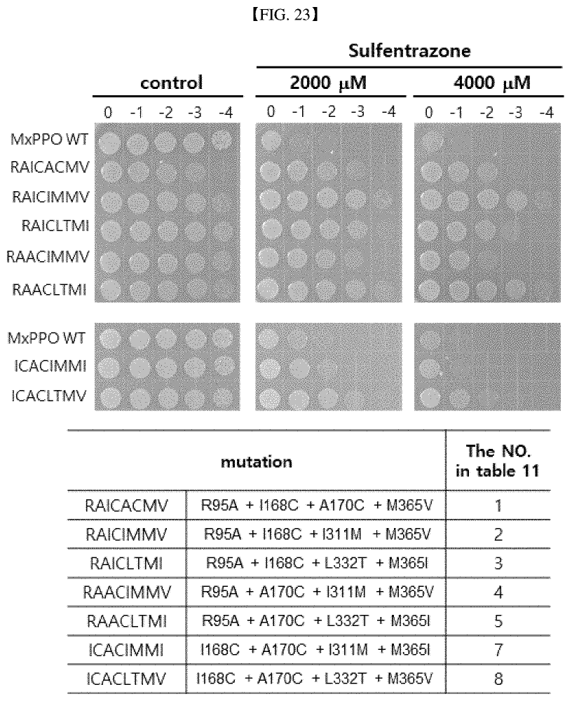

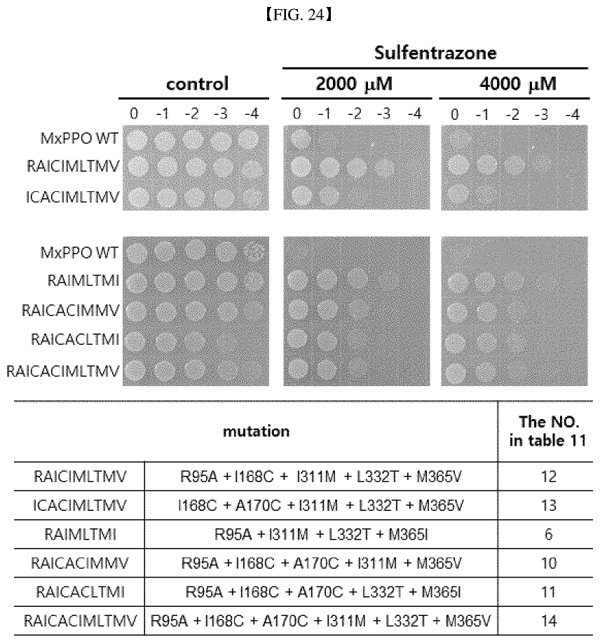

The LB agar media (LB 25 g/l, Bacto agar 15 g/l) containing carbenicillin (100 μg/ml) and 0 to 4,000 μM of various herbicides dissolved in DMSO was prepared. Next, 10 μl of each diluted solution was dropped on the plate and cultured at 37° C. under light (Tables 7, 9 and 10, FIGS. 2 to 6, 13 to 20) or dark (Tables 8 and 11, FIGS. 7 to 12, 21 to 24) for 16 to 20 hours. Then, the extent of tolerance was evaluated. PPO-inhibiting herbicides used in the experiments were listed in Table 6:

TABLE 6
PPO-inhibiting herbicides used in the experiments

| Family | Herbicide |
|---|---|
| Pyrimidinedione | tiafenacil |
| | saflufenacil |
| Diphenyl ether | fomesafen |
| | acifluorfen |
| N-phenylphthalimides | flumioxazin |
| Triazolinones | sulfentrazone |
| Oxazolidinediones | pentoxazone |
| Phenylpyrazoles | pyraflufen-ethyl |
| Others | pyraclonil |

The extent of herbicide tolerance of the ApPPO1 or MxPPO mutated genes was evaluated by comparing that of mutated genes with that of ApPPO1 or MxPPO wild type. The relative tolerance was represented with “+” as a factor of 10 times. Evaluation result was listed in Tables 7 to 11 and FIGS. 2 to 24

TABLE 7
Herbicide tolerance evaluation of mutated ApPPO1

| | Mutation | | | | | |
|---|---|---|---|---|---|---|
| No. | site | tiafenacil | saflufenacil | flumioxazin | sulfentrazone | Fomesafen |
| 1 | A215C | + | + | N.T | + | + |
| | (AC) | | | | | |
| 2 | A215L | ++ | ++++ | ++++ | +++ | +++ |
| | (AL) | | | | | |
| 3 | V360M | ++ | +++ | N.T | + | + |
| | (VM) | | | | | |
| 4 | Y422T | ++ | ++++ | +++ | + | + |
| | (YT) | | | | | |
| 5 | Y422C | ++ | +++ | N.T | ++ | ++ |
| | (YC) | | | | | |
| 6 | Y422M | +++ | +++++ | +++ | + | + |
| | (YM) | | | | | |
| 7 | Y422I (YI) | ++ | +++++ | +++ | + | + |
| 8 | Y422L | ++ | ++++ | +++ | + | + |
| | (YL) | | | | | |
| | WT | − | − | − | − | − |

| | Mutation | | | | |
|---|---|---|---|---|---|
| No. | site | acifluorfen | pyraclonil | pentoxazone | pyraflufenethyl |
| 1 | A215C | ++ | + | + | ++ |
| | (AC) | | | | |
| 2 | A215L | +++ | ++ | ++ | +++ |
| | (AL) | | | | |
| 3 | V360M | ++ | + | + | ++ |
| | (VM) | | | | |
| 4 | Y422T | + | + | ++ | ++ |
| | (YT) | | | | |
| 5 | Y422C | ++ | ++ | +++ | +++ |
| | (YC) | | | | |
| 6 | Y422M | ++ | ++ | ++ | ++ |
| | (YM) | | | | |
| 7 | Y422I (YI) | + | + | ++ | ++ |
| 8 | Y422L | ++ | ++ | ++ | ++ |
| | (YL) | | | | |
| | WT | − | − | − | − |

N.T (Not tested)

TABLE 8
Herbicide tolerance evaluation of mutated ApPPO1

| | | | flumiox- | sulfen- |
|---|---|---|---|---|
| No. | Mutation site | tiafenacil | azin | trazone |
| 1 | R140A + Y422I | +++++ | +++++ | ++++ |
| 2 | R140A + Y422T | ++++ | ++++ | +++ |
| 3 | R140A + Y422M | ++++ | ++++ | ++++ |
| 4 | V213C + Y422I | ++++ | +++++ | +++ |
| 5 | V213C + Y422T | +++++ | +++++ | ++++ |
| 6 | V213C + Y422M | +++++ | +++ | ++ |
| 7 | A215L + Y422I | ++++ | ++++ | ++++ |
| 8 | A215L + Y422T | + | + | ++++ |
| 9 | A215L + Y422M | +++++ | +++++ | ++++ |
| 10 | A215C + Y422I | +++++ | +++++ | ++++ |
| 11 | A215C + Y422T | +++ | +++ | +++ |
| 12 | A215C + Y422M | +++++ | +++++ | ++++ |
| 13 | R140A + V213C + | +++++ | +++++ | ++++ |
| | Y422I | | | |
| 14 | R140A + V213C + | +++++ | +++++ | ++++ |
| | Y422M | | | |
| 15 | R140A + A215C + | +++++ | +++++ | ++++ |
| | Y422I | | | |
| 16 | R140A + A215L + | +++++ | +++++ | ++++ |
| | Y422M | | | |
| 17 | V213C + A215C + | +++++ | +++++ | ++++ |
| | Y422I | | | |
| 18 | V213C + A215L + | +++++ | +++++ | ++++ |
| | Y422M | | | |
| 19 | R140A + V213C + | +++++ | +++++ | ++++ |
| | A215C + Y422I | | | |
| 20 | R140A + V213C + | +++++ | +++++ | ++++ |
| | A215L + Y422M | | | |
| | WT | − | − | − |

TABLE 9
Herbicide tolerance evaluation of mutated MxPPO

| | | | | flumiox- |
|---|---|---|---|---|
| No. | Mutation site | tiafenacil | saflufenacil | azin |
| 1 | A170C | + | +++ | + |
| 2 | A170L | + | ++ | ++ |
| 3 | I311M | ++ | ++ | ++ |
| 4 | M365I | + | ++ | ++ |
| 5 | M365L | + | ++ | ++ |
| 6 | M365V | + | +++ | ++ |
| | WT | − | − | − |

TABLE 10
Herbicide tolerance evaluation of mutated MxPPO

| | | | flumiox- | sulfen- |
|---|---|---|---|---|
| No. | Mutation site | tiafenacil | azin | trazone |
| 1 | R95A + M365I | N.T | + | + |
| 2 | R95A + M365V | N.T | + | + |
| 3 | I168C + M365I | N.T | + | + |
| 4 | I168C + M365V | N.T | + | + |
| 5 | A170C + M365I | N.T | + | + |
| 6 | A170C + M365V | + | ++ | + |
| 7 | I311M + M365I | + | ++ | + |
| 8 | I311M + M365V | + | ++ | + |
| 9 | L332T + M365I | N.T | + | + |
| 10 | L332T + M365V | + | + | + |
| 11 | R95A + I168C + M365I | N.T | + | + |
| 12 | R95A + I168C + M365V | N.T | + | + |
| 13 | R95A + A170C + M365I | N.T | + | + |
| 14 | R95A + I311M + M365I | + | ++ | + |
| 15 | R95A + I311M + M365V | N.T | ++ | + |
| 16 | R95A + L332T + M365I | N.T | + | + |
| 17 | R95A + L332T + M365V | N.T | + | + |
| 18 | I168C + A170C + M365V | N.T | ++ | ++ |
| 19 | I168C + I311M + M365I | + | ++ | + |
| 20 | I168C + I311M + M365V | + | ++ | + |
| 21 | I168C + L332T + M365I | N.T | ++ | ++ |
| 22 | I168C + L332T + M365V | + | +++ | ++ |
| 23 | A170C + I311M + M365I | N.T | ++ | + |
| 24 | A170C + L332T + M365V | N.T | ++ | ++ |
| 25 | I311M + L332T + M365I | + | ++ | ++ |
| 26 | I311M + L332T + M365V | + | +++ | ++ |
| | WT | − | − | − |

N.T (Not tested)

TABLE 11
Herbicide tolerance evaluation of mutated MxPPO

| | | flumiox- | sulfen- |
|---|---|---|---|
| No. | Mutation site | azin | trazone |
| 1 | R95A + I168C + A170C + M365V | + | ++ |
| 2 | R95A + I168C + I311M + M365V | + | +++ |
| 3 | R95A + I168C + L332T + M365I | + | ++ |
| 4 | R95A + A170C + I311M + M365V | + | + |
| 5 | R95A + A170C + L332T + M365I | + | +++ |
| 6 | R95A + I311M + L332T + M365I | + | +++ |
| 7 | I168C + A170C + I311M + M365I | + | + |
| 8 | I168C + A170C + L332T + M365V | + | ++ |
| 9 | A170C + I311M + L332T + M365I | + | N.T |
| 10 | R95A + I168C + A170C + I311M + | + | +++ |
| | M365V | | |
| 11 | R95A + I168C + A170C + L332T + | + | +++ |
| | M365I | | |
| 12 | R95A + I168C + I311M + L332T + | + | +++ |
| | M365V | | |
| 13 | I168C + A170C + I311M + L332T + | + | ++ |
| | M365V | | |
| 14 | R95A + I168C + A170C + I311M + | + | +++ |
| | L332T + M365V | | |
| | WT | − | − |

N.T (Not tested)

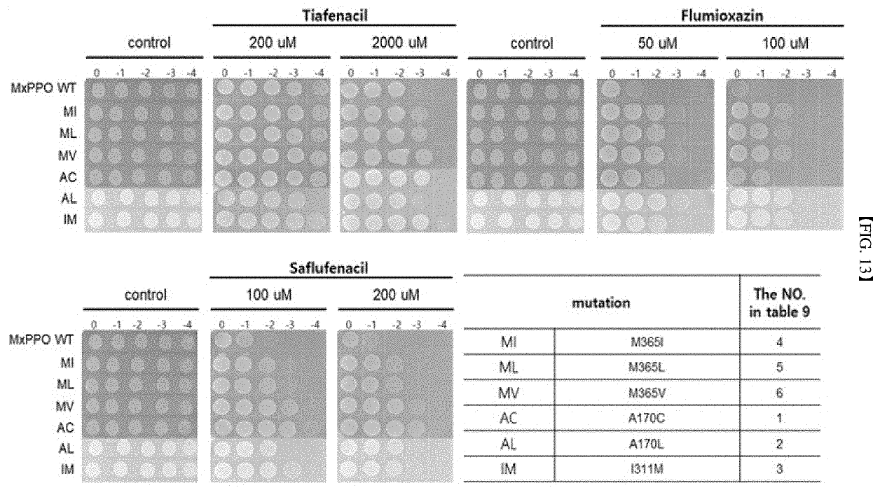

In Tables 7 to 11, tolerance level was presented as ‘−’ of tolerance of wild type and of variants equivalent to that of wild type, and was done as ‘+’ per each 10 fold resistance until ‘+++++’ as maximal resistance. (Tolerance level was evaluated by relative growth level of variants to that of wild type in the media containing highest concentration of herbicide; ‘+’=1-9 fold higher tolerance, ‘++’=10-99 fold higher tolerance, ‘+++’=100-999 fold higher tolerance, ‘++++’=1,000-9,999 fold higher tolerance, ‘+++++’=more than 10,000 fold higher tolerance) FIGS. 2 to 12 show the tolerance of ApPPO1 wild type and its variants, and FIGS. 13 to 24 show that of MxPPO wild type and its variants. The concentrations of herbicides were written on the photographs of tolerance test. A dilution series (OD₆₀₀=0.5, 0.05, 0.005, 0.0005, 0.00005) was made and spotted on LB agar plates supplemented with herbicides.

As shown in Tables 7 to 11 and FIGS. 2 to 24, all of BT3 strains transformed with variants of ApPPO1 or MxPPO showed higher tolerance level than that of wild type against various PPO-inhibiting herbicides.

### Example 4: Measurement of PPO Enzyme Activity and IC₅₀ Value for Herbicides

The enzyme activities of variants wherein amino acids of certain position of PPO protein mutated were measured and inhibition assay with the PPO-inhibiting herbicides was conducted.

Although the solubility of PPO protein is markedly low in aqueous condition, it was greatly increased when maltose binding protein (MBP) was fused to PPO protein. Thus, PPO proteins of wild type and variants were expressed as fused to MBP and were used for experiments.

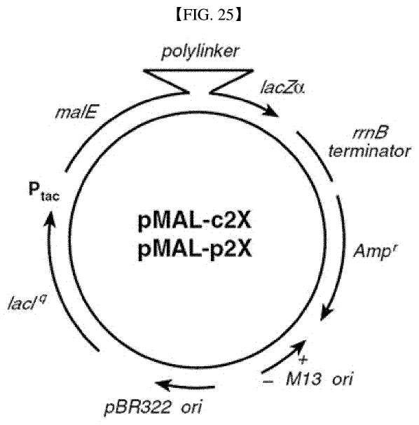

In order to express wild type and variant proteins of ApPPO1 and MxPPO, those genes were introduced into pMAL-c2x vector (refer to FIG. 25), respectively.

Detailed experimental procedure was as follows:

Using primers listed in Table 13, PCR was carried out to amplify PPO genes under following condition.

PCR reaction mixture

Template (synthetic DNA of ApPPO1 or MxPPO) 1 μl

10× buffer 5 μl

dNTP mixture (10 mM each) 1 μl

Forward primer (10 μM) 1 μl

Reverse primer (10 μM) 1 μl

DDW 40 μl

Pfu-X (Solgent, 2.5 units/μl) 1 μl

Total 50 μl

TABLE 12
PCR reaction condition

| | | | | |
|---|---|---|---|---|
| 94° C. | 4 | min. | 1 | cycle |
| 94° C. | 30 | sec. | 27 | cycles |
| 56° C. | 30 | sec. | | |
| 72° C. | 5 | min. | | |
| 72° C. | 5 | min. | 1 | cycle |
| 4° C. | 5 | min. | 1 | cycle |

TABLE 13
Primer list for cloning of ApPPO1
and MxPPO in pMAL-c2x

| | | | SEQ |
|---|---|---|---|
| | | | ID |
| Strain | Primer | Sequence | NO |
| Auxenochlorella | ApPPO1_ | CCCCGGATC | 83 |
| protothecoides | BamHIF | CATGGCCGA | |
| | | GTACGACGT | |
| | | TGT | |
| | ApPPO1_ | CCCCGTCGA | 84 |
| | SalIR | CTCAGGTTG | |
| | | CCAGACTTT | |
| | | TAACGT | |
| Myxococcus | MxPPO_ | CCCCGGATC | 85 |
| xanthus | BamHIF | CATGCACCA | |
| | | TATGCCCCG | |
| | | AAC | |
| | MxPPO_ | CCCCGTCGA | 86 |
| | SalIR | CTCAAGGCG | |
| | | CGTGTGATG | |
| | | TATTAC | |

Amplified PCR products and pMAL-c2x vector (NEB, FIG. 25) were digested with BamHI and SalI restriction enzymes, and ligated to construct pMAL-c2x-ApPPO1 and pMAL-c2x-MxPPO plasmids using T4 DNA ligase (RBC, 3 units/μl).

ApPPO1 and MxPPO genes cloned in pMAL-c2x vector were mutated through site-directed mutagenesis using primers listed in Tables 4 and 5, respectively.

PCR reaction mixture

Template 1 μl

10× buffer 5 μl

dNTP mixture (10 mM each) 1 μl

Forward primer (10 μM) 1 μl

Reverse primer (10 μM) 1 μl

DDW 40 μl

Pfu-X (Solgent, 2.5 units/μl) 1 μl

Total 50 μl

Then, BL21 CodonPlus(DE3) E. coli was transformed with constructs.

The transformed E. coli were cultured under the following conditions to express PPO proteins:

Induction: OD₆₀₀=0.2, addition of IPTG to 0.3 mM final concentration;

Culture temperature: 23° C., 200 rpm shaking culture;

Culture time: 16 hrs;

Culture volume: 200 ml/1,000 ml flask.

After harvesting the cells, cell lysis and protein extraction were performed by the following process:

Extraction buffer: Column buffer (50 mM Tris-Cl, pH 8.0, 200 mM NaCl) 5 ml buffer/g cell;

Sonication: SONICS&MATERIALS VCX130 (130 watts);

15 sec ON, 10 sec OFF for 5 min on ice;

Centrifugation at 4° C. for 20 minutes (20,000×g); and the supernatant obtained after the centrifugation was diluted at the ratio of 1:6 with column buffer.

The following process for purification of PPO protein was performed in a 4° C. cold room. Amylose resin (NEB) was packed to 1.5×15 cm column (Bio-Rad, Econo Columns 1.5×15 cm, glass chromatography column, max. vol), and the obtained protein extracts were loaded to the column at a flow rate of 0.2 ml/min. The column was washed with 3 column volumes of buffer and the presence of protein in the washing solution was examined. When the protein was no longer detected, the washing procedure was terminated. Then, the MBP-PPO protein was eluted with approximately 2 column volumes of buffer containing 20 mM maltose. The protein concentration of each eluent was determined and the elution was stopped when the protein was no longer detected. Ten microliter of each fraction was investigated for protein quantification and SDS-PAGE analysis. The highly pure fractions of PPO protein variants were used for the enzyme assay.

Since protoporphyrinogen IX, a substrate of PPO protein, was not commercially available, it was chemically synthesized in the laboratory. Overall process was performed in dark under nitrogen stream. Nine micrograms of protoporphyrin IX was dissolved in 20 ml of 20% (v/v) EtOH, and stirred under dark condition for 30 minutes. The obtained protoporphyrin IX solution was put into a 15 ml screw tube in an amount of 800 μl, and flushed with nitrogen gas for 5 minutes. To this, 1.5 g of sodium amalgam was added and vigorously shaken for 2 minutes. The lid was opened to exhaust hydrogen gas in the tube. Thereafter, the lid was closed and incubated for 3 minutes. The protoporphyrinogen IX solution was filtered using syringe and cellulose membrane filter. To 600 μl of the obtained protoporphyrinogen IX solution, approximately 300 μl of 2M MOPS [3-(N-morpholino) propanesulfonic acid] was added to adjust pH to 8.0. To determine the enzyme activity of PPO protein, a reaction mixture was prepared with the following composition (based on 10 ml): 50 mM Tris-Cl (pH 8.0); 50 mM NaCl; 0.04% (v/v) Tween 20; 40 mM glucose (0.072 g); 5 units glucose oxidase (16.6 mg); and 10 units catalase (1 μl).

Hundred and eighty microliters of a reaction mixture containing the purified PPO protein were placed in 96 well plates and 20 μl of purified PPO proteins were added. After 50 μl of the mineral oil was layered, the reaction was initiated by adding the substrate, protoporphyrinogen IX solution, to a final concentration of 50 μM. The reaction proceeded at room temperature for 30 min and the fluorescence of protoporphyrin IX was measured using Microplate reader (Sense, Hidex) (excitation: 405 nm; emission: 633 nm). To calculate the PPO enzyme activity, the protoporphyrinogen IX solution was kept open in the air overnight to oxidize the solution. To this, 2.7 N HCl was added, and the absorbance at 408 nm was measured. A standard curve was generated using standard protoporphyrin IX, and PPO activity was measured by calibration of protoporphyrin IX using the standard curve of protoporphyrin IX.

The enzyme activities of the obtained PPO wild type and variants were shown in Tables 14 to 15. Activities of variants were presented relatively compared to that of wild type.

The concentration of the PPO-inhibiting herbicides that inhibits the PPO enzyme activity of each PPO wild type and variants by 50% (IC₅₀) was measured for each herbicide. The final concentrations of each herbicide were as follows:
tiafenacil, flumioxazin and sulfentrazone: 0, 10, 50, 100, 250, 500, 1000, 2500, 5000, 10000 nM

The IC₅₀ value, the concentration of the herbicide inhibiting the PPO enzyme activity to 50%, was calculated by adding the herbicide of the above concentrations.

The IC₅₀ value for each herbicide was shown in the following Tables 14 and 15.

TABLE 14
Determination of IC₅₀ of ApPPO1 wild type
and mutants against various herbicides

| | | | | flumiox- | sulfen- |
|---|---|---|---|---|---|
| | | Activity | tiafenacil | azin | trazone |
| No. | Mutation site | (%) | (nM) | (nM) | (nM) |
| 1 | WT | 100 | 21 | 89 | 348 |
| 2 | R140A | 88 | 86 | 202 | 973 |
| 3 | F209A | 78 | 69 | N.T | N.T |
| 4 | V213C | 85 | 81 | 163 | 526 |
| 5 | A215C | 89 | 76 | N.T | N.T |
| 6 | A215L | 76 | 3,456 | 1,552 | >10,000 |
| 7 | V360M | 59 | 75 | N.T | N.T |
| 8 | F386V | 86 | 368 | N.T | N.T |
| 9 | L389T | 11 | 716 | N.T | N.T |
| 10 | I402T | 16 | 488 | N.T | N.T |
| 11 | Y422M | 93 | 457 | 237 | 1,084 |
| 12 | Y422I | 91 | 2,974 | 911 | 1,496 |
| 13 | Y422T | 84 | 3,660 | 935 | 3,778 |
| 14 | R140A + Y422M | 29 | 1,564 | 332 | 1,977 |
| 15 | F209A + Y422M | 51 | 699 | N.T | N.T |
| 16 | V213C + Y422M | 29 | 840 | 363 | 1,732 |
| 17 | A215C + Y422M | 58 | 3,541 | N.T | N.T |
| 18 | A215L + Y422M | 34 | >5,000 | >5,000 | >10,000 |
| 19 | V360M + Y422M | 8 | 1,162 | N.T | N.T |
| 20 | F386V + Y422M | 65 | 756 | N.T | N.T |
| 21 | L389T + Y422M | 15 | 1,956 | N.T | N.T |
| 22 | I402T + Y422M | 21 | 4,187 | N.T | N.T |
| 23 | V360I + Y422I | 16 | 3,282 | N.T | N.T |
| 24 | S362V + Y422I | 21 | 4,836 | N.T | N.T |

N.T (Not tested)

TABLE 15
Determination of IC₅₀ of MxPPO wild type
and mutants against various herbicides

| | | | | flumiox- | sulfen- |
|---|---|---|---|---|---|
| | | Activity | tiafenacil | azin | trazone |
| No. | Mutation site | (%) | (nM) | (nM) | (nM) |
| 1 | WT | 100 | 242 | 24 | 534 |
| 2 | R95A | 43 | 2,366 | 154 | >10,000 |
| 3 | V164A | 75 | 367 | N.T | N.T |
| 4 | I168C | 47 | 550 | 80 | 1,162 |
| 5 | A170C | 86 | 1,684 | 546 | 4,571 |
| 6 | A170L | 40 | >5,000 | >5,000 | >10,000 |
| 7 | I311M | 87 | 964 | 58 | 1,228 |
| 8 | F329V | 91 | 239 | N.T | N.T |
| 9 | L332T | 87 | 1,005 | 78 | 4,769 |
| 10 | I345T | 33 | 2,206 | N.T | N.T |
| 11 | M365I | 82 | 1,379 | 1,327 | 3,388 |
| 12 | M365V | 77 | 1,980 | 1,593 | 3,590 |
| 13 | M365T | 52 | 2,772 | N.T | N.T |
| 14 | R95A + M365I | 42 | >5,000 | N.T | N.T |
| 15 | R95A + M365V | 40 | >5,000 | N.T | N.T |
| 16 | I168C + M365I | 45 | 1,677 | N.T | N.T |
| 17 | I168C + M365V | 42 | 2,031 | N.T | N.T |
| 18 | A170C + M365I | 78 | 2,449 | 1,848 | >10,000 |
| 19 | A170C + M365V | 71 | 2,794 | N.T | N.T |
| 20 | A170L + M365I | 33 | >5,000 | N.T | N.T |
| 21 | A170L + M365V | 40 | >5,000 | N.T | N.T |
| 22 | I311M + M365I | 75 | 3,327 | N.T | N.T |
| 23 | I311M + M365V | 71 | 3,368 | N.T | N.T |
| 24 | L332T + M365I | 80 | 2,857 | N.T | N.T |
| 25 | L332T + M365V | 68 | 2,591 | N.T | N.T |
| 26 | R95A + I168C + | 38 | 3,982 | N.T | N.T |
| | M365I | | | | |
| 27 | R95A + A170C + | 41 | >5,000 | N.T | N.T |
| | M365I | | | | |
| 28 | R95A + I311M + | 37 | >5,000 | N.T | N.T |
| | M365V | | | | |
| 29 | R95A + L332T + | 38 | >5,000 | N.T | N.T |
| | M365I | | | | |
| 30 | I168C + A170C + | 45 | 3,577 | N.T | N.T |
| | M365V | | | | |
| 31 | I168C + I311M + | 47 | 4,671 | N.T | N.T |
| | M365I | | | | |
| 32 | I168C + L332T + | 49 | 3,196 | N.T | N.T |
| | M365V | | | | |
| 33 | A170C + I311M + | 69 | 4,572 | N.T | N.T |
| | M365I | | | | |
| 34 | I311M + L332T + | 55 | >5,000 | N.T | N.T |
| | M365V | | | | |
| 35 | R95A + I168C + | 33 | >5,000 | 2,477 | >10,000 |
| | A170C + M365I | | | | |
| 36 | R95A + A170C + | 31 | >5,000 | N.T | N.T |
| | I311M + M365V | | | | |
| 37 | R95A + A170C + | 35 | >5,000 | N.T | N.T |
| | L332T + M365I | | | | |
| 38 | R95A + I168C + | 37 | >5,000 | 1,891 | >10,000 |
| | I311M + M365V | | | | |
| 39 | R95A + I168C + | 34 | >5,000 | 2,368 | >10,000 |
| | L332T + M365I | | | | |
| 40 | R95A + I311M + | 29 | >5,000 | 2,996 | >10,000 |
| | L332T + M365V | | | | |
| 41 | I168C + A170C + | 44 | >5,000 | N.T | N.T |
| | I311M + M365I | | | | |
| 42 | I168C + A170C + | 40 | 4,537 | N.T | N.T |
| | L332T + M365V | | | | |
| 43 | A170C + I311M + | 52 | >5,000 | 3,627 | >10,000 |
| | L332T + M365I | | | | |
| 44 | R95A + I168C + | 17 | >5,000 | N.T | N.T |
| | A170C + I311M + | | | | |
| | M365V | | | | |
| 45 | R95A + I168C + | 18 | >5,000 | N.T | N.T |
| | A170C + L332T + | | | | |
| | M365I | | | | |
| 46 | R95A + I168C + | 12 | >5,000 | 3,741 | >10,000 |
| | I311M + L332T + | | | | |
| | M365V | | | | |
| 47 | I168C + A170C + | 20 | >5,000 | N.T | N.T |
| | I311M + L332T + | | | | |
| | M365V | | | | |
| 48 | R95A + I168C + | 8 | >5,000 | >5,000 | >10,000 |
| | A170C + I311M + | | | | |
| | L332T + M365V | | | | |

N.T (Not tested)

As shown in the Tables 14 and 15, it was demonstrated that variants of ApPPO1 and MxPPO proteins showed the significantly increased IC₅₀ values against each herbicide compared to the wild type. Such results indicate that herbicide tolerance was increased by amino acid substitutions at specified positions of PPO protein. Although the data showed that ApPPO1 and MxPPO protein variants possess reduced enzyme activity compared to the wild type, it might be caused by the difference between the chloroplast environment where PPO functions and in vitro assay condition. Thus, when PPO variants are properly assembled and expressed to chloroplasts in plants, the enzyme activity would not be affected drastically.

### Example 5. Generation of Arabidopsis thaliana Transformants Using ApPPO1 or MxPPO Variants and PPO-Inhibiting Herbicide Tolerance Test

5-1. Construction of A. thaliana Transformation Vectors and Generation of A. thaliana Transformants

A. thaliana was transformed with a binary vector having ORF of a selectable marker, Bar gene (glufosinate-tolerant gene), and ORF of each gene of ApPPO1 variants, MxPPO, and MxPPO variants. The transgenic plant was examined for cross-tolerance towards glufosinate and PPO-inhibiting herbicides. The bar gene was also used to examine whether the transgene was stably inherited during generations. NOS promoter and E9 terminator were used for bar gene expression.

In order to express proteins of ApPPO1 variants, MxPPO, and MxPPO variants in plants, a CaMV35S promoter and a NOS terminator were used. Encoding genes of ApPPO1 variants, MxPPO, and MxPPO variants were introduced into binary vector using XhoI and BamHI restriction enzymes. Furthermore, for confirmation of the protein expression, hemagglutinin (HA) tag was fused to the C-terminal region of PPO protein coding gene using BamHI and SacI restriction enzymes. In addition, in order to transit protein to chloroplast, transit peptide (TP) coding gene (SEQ ID NO: 2) of AtPPO1 gene (SEQ ID NO: 87) was fused to N-terminal region of PPO protein coding gene using XbaI and XhoI restriction enzymes.

Each constructed vector was transformed to Agrobacterium tumefaciens GV3101 competent cell by freeze-thaw method. Agrobacterium GV3101 competent cells were prepared by following procedures, Agrobacterium GV3101 strain was cultured in 5 ml LB media at 30° C., 200 rpm for 12 hrs. The cells were subcultured in 200 ml of LB media at 30° C., 200 rpm for 3 to 4 hrs, and centrifuged at 3,000×g at 4° C. for 20 minutes. The cell pellet was washed with sterile distilled water, and then resuspended in 20 ml of LB media. Snap frozen 200 μl aliquots with liquid nitrogen were stored in a deep freezer.

Each transformed Agrobacterium was screened in spectinomycin-containing LB media. The screened colony was cultured in LB broth. After Agrobacterium cell was harvested from the culture media, it was resuspended in the solution containing 5% sucrose (w/v) and 0.05% Silwet L-77 (v/v) (Momentive Performance Materials Co., Ltd.) at an absorbance (OD₆₀₀) of 0.8. By floral dipping method, A. thaliana wild type (Col-0 ecotype) was transformed, and then the T₁ seeds were harvested after 1 to 2 months.

Transgenic plants were screened with glufosinate tolerance which was conferred by Bar gene expression in the binary vector. The obtained T₁ seeds were sown in ½ MS media (2.25 g/l MS salt, 10 g/l sucrose, 7 g/l Agar) supplemented with 50 μM glufosinate, and the surviving plants were selected 7 days after sowing. They were, then, transplanted into soil and grown to obtain T₁ plants.

In order to examine PPO-inhibiting herbicide tolerance of the transgenic plants, 4-week-old plants were evenly sprayed with herbicide (100 ml of 1 μM tiafenacil and 0.05% Silwet L-77 (v/v)) in 40×60 cm area (0.24 m²). While wild type A. thaliana (Col-0 ecotype) completely died within 7 days after treatment, each transgenic plant showed no damage to PPO-inhibiting herbicide treatment.

The T₂ seeds were harvested from T₁ transgenic plants and were sown to ½ MS media (2.25 g/l MS salt, 10 g/l sucrose, 7 g/l Agar) supplemented with 50 μM glufosinate. One week later, surviving plants were transplanted to soil.

5-2. Verification of Herbicide Tolerance of Transformed Arabidopsis Plants (T₂) Arabidopsis plants (T₂) transformed with a gene encoding an ApPPO1 variant (Y422I, Y422L, Y422M, Y422V, or A215L+Y422M), MxPPO, or a MxPPO variant (M365I) were tested for their tolerance against herbicides.

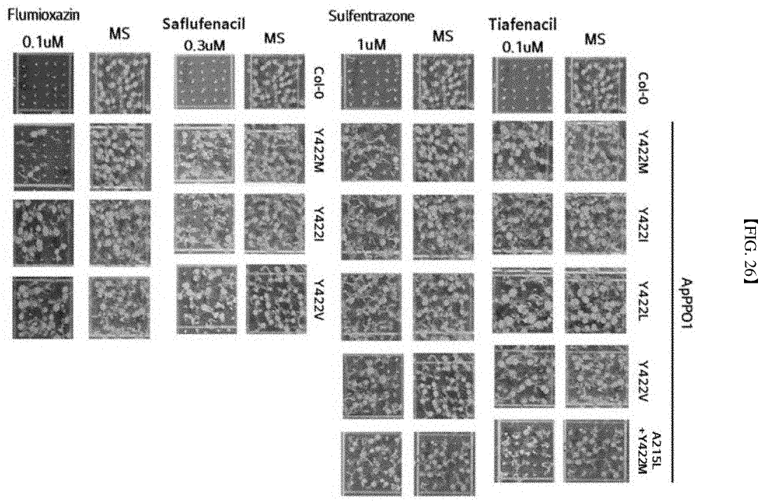

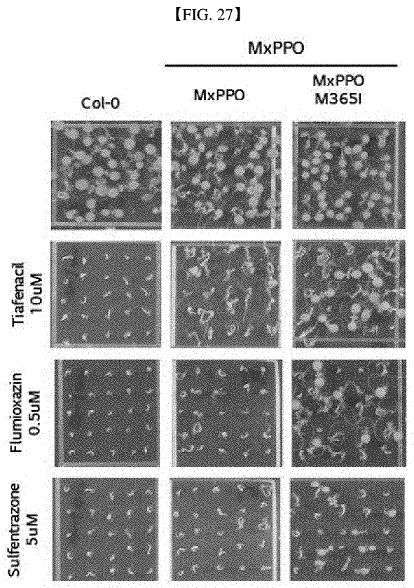

The T₂ seeds of ApPPO1 transgenic plants transformed with a gene encoding each of ApPPO1 variant (Y422I, Y422L, Y422M, Y422V, or A215L+Y422M), MxPPO, or a MxPPO variant (M365I) were sown to ½ MS media containing herbicide. Six days later, the extent of germination of each seeds was evaluated. A wild type A. thaliana (Col-0 ecotype) was used as a control. The obtained results are shown in FIG. 26 (ApPPO1 variant) and FIG. 27 (MxPPO wild type and MxPPO variant).

The concentrations of herbicide used are as follows:

FIG. 26: 0.1 μM tiafenacil, 0.3 μM saflufenacil, 0.1 μM flumioxazin, and 1 μM sulfentrazone, respectively; and

FIG. 27: 10 μM tiafenacil, 0.5 μM flumioxazin, and 5 μM sulfentrazone, respectively.

The seeds of wild type A. thaliana (Col-0 ecotype) germinated well in herbicide-free media, but did not normally germinate in herbicide-containing media as above. FIG. 26 demonstrates that each seeds of transgenic plants of ApPPO1 variants show excellent germinated rate and survival rate compared to those of the control Col-0. FIG. 27 demonstrates that each seeds of transgenic plants of MxPPO variants show excellent germinated rate and survival rate compared to those of the control Col-0 and MxPPO wild type.

1. A polypeptide selected from the group consisting of:
a polypeptide comprising an amino acid sequence of modified SEQ ID NO: 1, wherein one or more amino acid residues selected from the group consisting of R140, F209, V213, A215, G216, V360, S362, F386, L389, L399, I402, and Y422 of the amino acid sequence of SEQ ID NO: 1 are respectively and independently deleted or substituted with an amino acid selected from the group consisting of M(Met), V(Val), I(Ile), T(Thr), L(Leu), C(Cys), A(Ala), S(Ser), F(Phe), P(Pro), W(Trp), N(Asn), Q(Gln), G(Gly), Y(Tyr), D(Asp), E(Glu), R(Arg), H(His), and K(Lys), which is different from the amino acid at the corresponding position of SEQ ID NO: 1;
a polypeptide comprising an amino acid sequence of modified SEQ ID NO: 3, wherein one or more amino acid residues selected from the group consisting of R95, V164, I168, A170, G171, I311, V313, F329, L332, L342, I345, and M365 of the amino acid sequence of SEQ ID NO: 3 are respectively and independently deleted or substituted with an amino acid selected from the group consisting of M(Met), V(Val), I(Ile), T(Thr), L(Leu), C(Cys), A(Ala), S(Ser), F(Phe), P(Pro), W(Trp), N(Asn), Q(Gln), G(Gly), Y(Tyr), D(Asp), E(Glu), R(Arg), H(His), and K(Lys), which is different from the amino acid at the corresponding position of SEQ ID NO: 3; and
a polypeptide comprising an amino acid sequence with at least 95% identity with the amino acid sequence of the polypeptide.

2. The polypeptide of claim 1, which is selected from the group consisting of:
a polypeptide comprising an amino acid sequence of modified SEQ ID NO: 1, wherein one or more amino acid residues selected from the group consisting of R140, F209, V213, A215, G216, V360, S362, F386, L389, L399, I402, and Y422 of the amino acid sequence of SEQ ID NO: 1 are respectively and independently deleted or substituted with an amino acid selected from the group consisting of M(Met), V(Val), I(Ile), T(Thr), L(Leu), C(Cys), S(Ser), and A(Ala), which is different from the amino acid at the corresponding position of SEQ ID NO: 1;
a polypeptide comprising an amino acid sequence of modified SEQ ID NO: 3, wherein one or more amino acid residues selected from the group consisting of R95, V164, I168, A170, G171, I311, V313, F329, L332, L342, I345, and M365 of the amino acid sequence of SEQ ID NO: 3 are respectively and independently deleted or substituted with an amino acid selected from the group consisting of M(Met), V(Val), I(Ile), T(Thr), L(Leu), C(Cys), S(Ser), and A(Ala), which is different from the amino acid at the corresponding position of SEQ ID NO: 3; and
a polypeptide comprising an amino acid sequence with at least 95% identity with the amino acid sequence of the polypeptide.

3. The polypeptide of claim 1, which is selected from the group consisting of:
a polypeptide comprising an amino acid sequence having modification to SEQ ID NO: 1, wherein the modification comprises at least one amino acid mutation selected from the group consisting of Y422M, Y422L, Y422C, Y422V, Y422I, Y422T, A215L, A215C, A215I, V360M, R140A, F209A, V213C, V213S, F386V, L389T, I402T, V360I, V360L, and S362V, in the amino acid sequence of SEQ ID NO: 1;
a polypeptide comprising an amino acid sequence having modification to SEQ ID NO: 3, wherein the modification comprises at least one amino acid mutation selected from the group consisting of M365T, M365L, M365C, M365V, M365I, R95A, V164A, I168C, I168S, A170C, A170L, A170I, I311M, F329V, L332T, and I345T, in the amino acid sequence of SEQ ID NO: 3;
a polypeptide comprising an amino acid sequence with at least 95% identity with the amino acid sequence of the polypeptide.

4. The polypeptide of claim 3, which is selected from the group consisting of:
(1) a polypeptide comprising an amino acid sequence having modification to SEQ ID NO: 1, wherein the modification is selected from the group consisting of amino acid mutations of Y422M, Y422L, Y422C, Y422V, Y422I, Y422T, A215L, A215C, A215I, V360M, R140A, F209A, V213C, V213S, F386V, L389T, I402T, V360I, V360L, S362V, R140A+Y422I, R140A+Y422T, R140A+Y422M, F209A+Y422M, V213C+Y422I, V213C+Y422T, V213C+Y422M, A215C+Y422I, A215C+Y422T, A215C+Y422M, A215L+Y422I, A215L+Y422T, A215L+Y422M, V360M+Y422M, F386V+Y422M, V360M+Y422I, L389T+Y422M, I402T+Y422M, V360I+Y422I, V360I+S362V, S362V+Y422I, R140A+V213C+Y422I, R140A+V213C+Y422M, R140A+A215C+Y422I, R140A+A215L+Y422M, V213C+A215C+Y422I, V213C+A215L+Y422M, V360I+S362V+Y422I, A215C+V360M+Y422M, A215L+V360M+Y422M, A215I+V360M+Y422M, V213C+A215C+Y422M, V213C+A215L+Y422M, R140A+V213C+A215C+Y422I, and R140A+V213C+A215L+Y422M, in the amino acid sequence of SEQ ID NO: 1;
a polypeptide comprising an amino acid sequence having modification to SEQ ID NO: 3, wherein the modification is selected from the group consisting of amino acid mutations of M365T, M365L, M365C, M365V, M365I, R95A, V164A, I168C, I168S, A170C, A170L, A170I, I311M, F329V, L332T, I345T, R95A+M365I, R95A+M365V, I168C+M365I, I168C+M365V, A170C+M365I, A170C+M365V, A170L+M365I, A170L+M365V, I311M+M365I, I311M+M365V, L332T+M365I, L332T+M365V, V164A+M365I, F329V+M365I, I345T+M365I, A170C+I311M, A170L+I311M, A170I+I311M, I168C+A170C, I168C+A170L, R95A+I168C+M365I, R95A+I168C+M365V, R95A+A170C+M365I, R95A+I311M+M365I, R95A+I311M+M365V, R95A+L332T+M365I, R95A+L332T+M365V, I168C+A170C+M365V, I168C+I311M+M365I, I168C+I311M+M365V, I168C+L332T+M365I, I168C+L332T+M365V, A170C+I311M+M365I, A170C+L332T+M365V, I311M+L332T+M365I, I311M+L332T+M365V, R95A+I168C+A170C+M365I, R95A+I168C+A170C+M365V, R95A+A170C+I311M+M365V, R95A+A170C+L332T+M365I, R95A+I168C+I311M+M365V, R95A+I168C+L332T+M365I, R95A+I311M+L332T+M365I, R95A+I311M+L332T+M365V, I168C+A170C+I311M+M365I, I168C+A170C+L332T+M365V, A170C+I311M+L332T+M365I, R95A+I168C+A170C+I311M+M365V, R95A+I168C+A170C+L332T+M365I, R95A+I168C+I311M+L332T+M365V, I168C+A170C+I311M+L332T+M365V, and R95A+I168C+A170C+I311M+L332T+M365V, in the amino acid sequence of SEQ ID NO: 3;
a polypeptide comprising an amino acid sequence with at least 95% identity with the amino acid sequence of the polypeptide.

5. A polynucleotide encoding the polypeptide of claim 1.

6. A recombinant vector comprising the polynucleotide of claim 5.

7. A recombinant cell comprising the recombinant vector of claim 6.

8. A composition for conferring or enhancing herbicide tolerance of a plant or algae, comprising one or more selected from the group consisting of:
the polypeptide of claim 1;
a polynucleotide encoding the polypeptide;
a recombinant vector comprising the polynucleotide; and
a recombinant cell comprising the recombinant vector.

9. The composition of claim 8, wherein the herbicide is an herbicide inhibiting protoporphyrinogen IX oxidase.

10. The composition of claim 8, wherein the herbicide is at least one selected from the group consisting of pyrimidinediones, diphenyl-ethers, phenylpyrazoles, N-phenylphthalimides, phenylesters, thiadiazoles, oxadiazoles, triazolinones, oxazolidinediones, pyraclonil, flufenpyr-ethyl, and profluazol.

11. The composition of claim 10, wherein the herbicide is at least one selected from the group consisting of butafenacil, saflufenacil, benzfendizone, tiafenacil, fomesafen, oxyfluorfen, aclonifen, acifluorfen, bifenox, ethoxyfen, lactofen, chlomethoxyfen, chlorintrofen, fluoroglycofen-ethyl, halosafen, pyraflufen-ethyl, fluazolate, flumioxazin, cinidon-ethyl, flumiclorac-pentyl, fluthiacet, thidiazimin, oxadiargyl, oxadiazon, carfentrazone, sulfentrazone, azafenidin, pentoxazone, pyraclonil, flufenpyr-ethyl, profluazol, phenopylate, carbamate analogues of phenopylate, and agriculturally acceptable salt thereof.

12. The composition of claim 8, wherein the plant or algae further comprise a second herbicide-tolerant polypeptide or a gene encoding the same, and its tolerance to the second herbicide is conferred or enhanced.

13. The composition of claim 12, wherein the second herbicide is selected from the group consisting of glyphosate, glufosinate, dicamba, 2,4-D(2,4-Dichlorophenoxyacetic acid), isoxaflutole, ALS(acetolactate synthase)-inhibiting herbicide, photosystem II-inhibiting herbicide, phenylurea-based herbicide, bromoxynil-based herbicide, and combinations thereof.

14. The composition of claim 12, wherein the second herbicide-tolerant polypeptide is one or more selected from the group consisting of:
glyphosate herbicide-tolerant EPSPS (glyphosate resistant 5-enolpyruvylshikimate-3-phosphate synthase), GOX (glyphosate oxidase), GAT (glyphosate-N-acetyltransferase) or glyphosate decarboxylase;
glufosinate herbicide-tolerant PAT (phosphinothricin-N-acetyltransferase);
dicamba herbicide-tolerant DMO (dicamba monooxygenase);
2,4-D (2,4-dichlorophenoxyacetic acid) herbicide-tolerant 2,4-D monooxygenase or AAD (aryloxyalkanoate dioxygenase);
ALS (acetolactate synthase)-inhibiting sulfonylurea-based herbicide-tolerant ALS (acetolactate synthase), AHAS (acetohydroxyacid synthase) or AtAHASL (Arabidopsis thaliana acetohydroxyacid synthase large subunit);
photosystem II-inhibiting herbicide-tolerant photosystem II protein D1;
phenylurea herbicide-tolerant Cytochrome P450;
plastid-inhibiting herbicide-tolerant HPPD (hydroxyphenylpyruvate dioxygenase);
bromoxynil herbicide-tolerant nitrilase; and
combinations thereof.

15. The composition of claim 12, wherein the gene encoding the second herbicide-tolerant polypeptide is one or more selected from the group consisting of:
glyphosate herbicide-tolerant cp4 epsps, mepsps, 2mepsps, goxv247, gat4601 or gat4621 gene;
glufosinate herbicide-tolerant BAR or PAT gene;
dicamba herbicide-tolerant dmo gene;
2,4-D(2,4-dichlorophenoxyacetic acid) herbicide-tolerant AAD-1 or AAD-12 gene;
isoxaflutole herbicide-tolerant HPPDPF W336 gene;
sulfonylurea herbicide-tolerant ALS, Csr1, Csr1-1, Csr1-2, GM-HRA, S4-HRA, Zm-HRA, SurA or SurB gene;
photosystem II-inhibiting herbicide-tolerant psbA gene;
phenylurea herbicide-tolerant CYP76B1 gene;
bromoxynil herbicide-tolerant bxn gene; and
combinations thereof.

16. A transformant of a plant or algae having herbicide tolerance, or a clone or progeny thereof, comprising the polypeptide of claim 1 or a polynucleotide encoding the same.

17. The transformant, clone, or progeny thereof of claim 16, wherein the transformant is an alga, or a cell, protoplast, callus, hypocotyl, seed, cotyledon, shoot, or whole body of a plant.

18. A method of preparing a transgenic plant or algae having herbicide tolerance, the method comprising introducing the the polypeptide of claim 1 or a polynucleotide encoding the same into an alga, or a cell, protoplast, callus, hypocotyl, seed, cotyledon, shoot, or whole body of a plant.

19. A method of conferring or enhancing herbicide tolerance of a plant or algae, the method comprising introducing the the polypeptide of claim 1 or a polynucleotide encoding the same into an alga, or a cell, protoplast, callus, hypocotyl, seed, cotyledon, shoot, or whole body of a plant.

20. A method of controlling weeds in a cropland, the method comprising:
providing the cropland with a plant comprising the polypeptide of claim 1 or a polynucleotide encoding the same, and
applying an effective dosage of protoporphyrinogen IX oxidase-inhibiting herbicide to the cropland or the plant.

21. The method of claim 20, wherein the step of applying an effective dosage of protoporphyrinogen IX oxidase-inhibiting herbicide to the cropland is performed by applying an effective dosage of two or more kinds of protoporphyrinogen IX oxidase-inhibiting herbicides sequentially or simultaneously.

22. The method of claim 20,
wherein the plant further comprises a second herbicide-tolerant polypeptide or a gene encoding the same, and
the step of applying an effective dosage of protoporphyrinogen IX oxidase-inhibiting herbicide to the cropland is performed by applying effective dosages of the protoporphyrinogen IX oxidase-inhibiting herbicide and a second herbicide are applied sequentially or simultaneously.

23. A method of removing an undesired aquatic organism from a culture media, the method comprising:
providing a culture media with algae comprising the polypeptide of claim 1 or a polynucleotide encoding the same, and
applying an effective dosage of protoporphyrinogen IX oxidase-inhibiting herbicide to the culture media.


SEQUENCE LISTING (87 sequences)

SEQ ID NO: 1 - protein, 532 aa, Artificial Sequence
  Synthetic (Protoporphyrinogen IX oxidase (ApPPO1) of Auxenochlorella protothecoides)
MPFTHLKPLNRLLGSPRARHPHVAPQAVSVTNTSTTRPGGRSSAPGIPGDAEYDVVIVGAGISGLTTAQALAKQYSDAVPRLLVTEARDCVGGNIITRRDGGYQWEEGPNSFQPSDAVLRAAVDAGVADRLVLGDPKAPRYVYWGGRLRATPSGPDVLTFDLLSLWGKLRAGLGAAGFKAPLPEREESVKEFVTRNLGEEVFQRLIEPFCSGVYAGDPAKLSMKAAFGKVYDLEKGGGSIVGGVLALMKARKQKPPEPRPADLPPKPKGQTVGSFDKGLVTLPSAIAEELGDRVVTGWKLESIDRSGDRFQLTYDTPQGRKKVSARSVALTVPAYTAASLLESRFPKLGKALSSIYYPPVASVALSYPETAILPERLGSDGHLAGFGQLHPRTQGVTTLGTIYASSLFPGRVPEGKVLLLSYFGGATNTRVATMPHGEIVSQVDKDLRKMLIRSDAESPHTVNVTVWPRAIPQFNVGHTDLIQEAKDDLASQGWQGLHLGGNYVAGVALGKCMEAGYKQAAELASHVKSLAT

SEQ ID NO: 2 - DNA, 155 bp, Artificial Sequence
  Synthetic (Gene for Transit Peptide of AtPPO1)
atggagttattcttctccgtccgacgactcaatcgcttcttccgtcgttttcgaagcccaatctccgattaaatgtttataagcctcttagactccgttgttcagtggccggtggaccaaccgtcggatcttcaaaaatcgaaggcggaggaggc

SEQ ID NO: 3 - protein, 471 aa, Artificial Sequence
  Synthetic (Protoporphyrinogen IX oxidase (MxPPO) of  Myxococcus xanthus)
MHHMPRTTGMNVAVVGGGISGLAVAHHLRSRGTDAVLLESSARLGGAVGTHALAGYLVEQGPNSFLDREPATRALAAALNLEGRIRAADPAAKRRYVYTRGRLRSVPASPPAFLASDILPLGARLRVAGELFSRRAPEGVDESLAAFGRRHLGHRATQVLLDAVQTGIYAGDVEQLSVAATFPMLVKMEREHRSLILGAIRAQKAQRQAALPAGTAPKLSGALSTFDGGLQVLIDALAASLGDAAHVGARVEGLAREDGGWRLIIEEHGRRAELSVAQVVLAAPAHATAKLLRPLDDALAALVAGIAYAPIAVVHLGFDAGTLPAPDGFGFLVPAEEQRRMLGAIHASTTFPFRAEGGRVLYSCMVGGARQPGLVEQDEDALAALAREELKALAGVTARPSFTRVFRWPLGIPQYNLGHLERVAAIDAALQRLPGLHLIGNAYKGVGLNDCIRNAAQLADALVAGNTSHAP

SEQ ID NO: 4 - DNA, 1416 bp, Artificial Sequence
  Synthetic (MxPPO coding gene)
atgcaccacatgccgaggacaactggaatgaatgtcgccgtcgtgggaggtgggatttcggggttggccgtcgcgcatcatttgcgctcgcgcggtacggatgccgtgcttctggagtcctccgcccgacttggcggcgcggtgggcacgcatgcgctcgccggctacctggtggagcaggggcccaacagcttcctggaccgcgagcccgcaacgcgggcgctcgcggcggcgctgaatctggaggggcgaattcgcgccgcggacccggcggcgaagcgtcgctatgtctacacgcggggcagactccggtcggtaccggcctcgccgcccgcgtttctcgcatcggacattctgccgctcggcgcgcggttgcgcgtcgctggcgaactgttctcccgccgcgcgccggagggtgtggacgaatcgctggccgccttcggccgccgccacctgggacacagggcgacgcaggtgctgctcgacgcggtgcagacgggcatctacgcgggcgacgtggagcagctcagcgtcgcggccaccttccccatgctggtgaagatggagcgcgagcaccgcagcctcatcctgggcgccatccgcgcgcagaaggctcagcgccaggcggcgctccccgcgggaacggcgccgaagttgagcggcgcgctgagcacgttcgacggcggattgcaggtgctcatcgacgcgctcgccgcatcgttgggtgacgcggcgcacgtgggcgcgcgcgtggaggggctggcgcgcgaggacggcggttggaggctcatcatcgaggagcacggacgccgcgcggaactgtccgtggcccaggtggtactggcggcgcccgcgcatgccaccgcgaaattgctacgcccactggatgacgcgctcgccgccctggtggcgggtatcgcctatgcgcccatcgcggtggtgcacctgggcttcgacgcggggacacttccggcgccggatggctttgggttcctggtgccagcggaggagcagcggcggatgctgggcgccatccacgcgtccaccacctttcccttccgggccgagggcggacgcgtgctctattcctgcatggtggggggcgcgcgtcagccagggctggtggaacaggacgaggatgcattggcggcgctggcgcgcgaggaactgaaggcgctggcaggcgtgacggcgcgcccctccttcacgcgggtgttccgctggccgcttggcattccccagtacaacctgggacacctggagcgcgtggccgctatcgacgcggcgctgcaacgcttgcccggcctgcacctcatcgggaatgcgtacaagggcgtgggcctcaacgactgcatccgcaacgcggcgcaactcgcggacgccctggtcgcggggaacacctcccacgccccgtag

SEQ ID NO: 5 - protein, 467 aa, Artificial Sequence
  Synthetic (Protoporphyrinogen IX oxidase (CyPPO10) of Thermosynechococcus elongatus BP-1)
MIEVDVAIVGGGLSGLSVAWRLQRSAPHYSGVLLEASDRLGGNITTQAAEGFVWELGPNSFAPTPALLQLIAEVGLHSELIRGDRHLPRYIYWRGELYPLEPTRPLALATSNLLSPWGKVRAALGALGFVPPYLGSGDESVDSFFRRHLGQEVAERLVAPFVSGVYAGDPQQLSAAAAFRRIAQLEKLGGSLIAGALRLRRQQPPQPKPPAQVQMRPGELGSFREGLAALPRAIAQQLKAPLHLQTPVEAITPEPKGGYLLRSGEQTWHARSVVLATPAYQTAELVAPFQPAIARALATIPYPTVACVVLAYPAGLGRSVRPGFGVLVPRGQGIRTLGTIWSSCLFPQRTPAGWQVFTSFIGGATDPDLASLREEAIVEQVQQDLTRLLDLPAAKARLLGMKVWRRAIPQYIVGYPQQWQQVTHALTQTPGLFLCSNYAEGVALGDRVEHGNRTAAAVAAYLAGGQS

SEQ ID NO: 6 - DNA, 1404 bp, Artificial Sequence
  Synthetic (CyPPO10 coding gene)
atgattgaagtggatgtggctattgttggtggtggtcttagtggattgtcagtggcttggagattacagaggagtgctcctcattattctggagttcttcttgaggcttctgatagacttggaggtaatatcactacacaagctgctgaaggatttgtgtgggagcttggtccaaacagtttcgctcctactccagcactcttacagttgattgctgaagttggactccattctgagttaatcagaggagataggcaccttccaagatatatatactggaggggagaactttatcctttggagccaactaggcctcttgctttggcaacatcaaatcttttgagtccttggggaaaggttagagctgcactcggagctttaggttttgtgcctccatatcttggatctggagatgaaagtgttgattctttctttagaaggcatcttggacaagaagttgctgagagattggtggcaccatttgtttcaggagtgtacgctggagatcctcaacagctttctgctgctgctgcttttagaaggattgctcaacttgagaagttgggaggttcattgatcgctggagcactcagattaagaaggcaacagcctccacagccaaaacctccagctcaagtgcagatgagacctggagaactcggtagttttagggagggtctcgctgcattacctagagctatcgcacaacagttgaaggcaccacttcatttgcaaacacctgttgaagctattacccctgagccaaaaggaggttatctcttaaggagtggtgaacagacttggcacgctagatcagttgtgttggctactccagcataccaaactgctgaacttgttgcaccattccagcctgctatcgctagagctttggctaccataccttatccaactgttgcttgtgttgtgcttgcttaccctgctggattgggtagatcagttagacctggatttggtgttttggtgcctagaggacaaggtataaggacactcggaaccatttggtcttcatgcttattcccacaaagaactcctgctggttggcaggtttttacctctttcataggaggtgctactgatcctgatcttgcatcattgagagaagaggctattgttgaacaagtgcaacaggatctcacaaggcttcttgatcttcctgctgcaaaggcaagactcttgggtatgaaggtttggagaagggctattccacaatatatcgttggttaccctcaacagtggcaacaggtgacacacgctcttacccagactcctggtctcttcttatgttcaaactacgctgagggagttgcattgggagatagagtggaacacggaaataggactgctgctgctgtggctgcttacctcgctggtggacaatcataa

SEQ ID NO: 7 - DNA, 1599 bp, Artificial Sequence
  Synthetic (Optimized codon encoding ApPPO1)
atgcccttcacgcacttaaaacccttgaaccggctgctgggtagccccagggctcggcatccgcatgtcgcaccccaggccgtctccgtcaccaacacgagcaccacgcggccgggcggcaggagctccgcgccagggatcccaggcgacgccgagtacgacgttgtgatcgtcggagccggtatctctgggttaacaacagcccaagcgctggcgaagcagtattctgatgctgtacctcgacttctagtgacagaagcacgtgactgcgtaggaggtaacatcataacgcgaagagacggaggataccagtgggaggaaggaccgaattcatttcagccctccgatgcggtattgagggctgccgtagacgcgggggtcgccgatcgtttggttctaggagatccgaaggcgcccaggtacgtttattggggtgggaggctccgagctacgcctagtgggccggatgttttgactttcgatctcctaagcctatgggggaaactgagagctggacttggcgccgcggggttcaaggcaccattgcccgaacgagaagagtctgtcaaagagttcgtcacgagaaatttaggagaggaggtattccaacgacttatagagccgttttgcagcggggtttacgccggcgacccggctaagttgagtatgaaggccgcttttggaaaagtttatgatctggagaaggggggcggatctattgtgggcggcgtactagctctgatgaaggcacgaaaacagaagccaccagagcctaggcccgctgaccttcctccgaagccgaagggtcaaactgtcggttcttttgataaaggattagtcacactcccttcagctattgccgaggaactgggcgaccgtgttgtaacaggctggaagttggaaagtatcgatagatcaggtgacagattccagttaacctacgacaccccccagggcagaaaaaaggtaagtgcacgtagcgtggctttaacagtcccggcatataccgcggcgtcactgctagagagtaggttccctaagctggggaaggcccttagctctatctattaccctcccgtcgcatctgtagcattatcttaccctgagacggcgatactccctgaaaggttagggagtgatggtcacctagcgggctttggccagctacaccctcgtactcagggtgtcaccactctgggcactatatatgcctcaagcttattcccgggacgtgttcccgagggaaaggtgctgctcttgtcatattttgggggggctaccaacacccgtgtcgccaccatgccccacggggaaatcgtttcccaagtagacaaggatctgcgtaaaatgttgatacgttccgatgcggaatccccccatactgtaaatgtaaccgtttggcctcgtgccataccacaatttaacgtgggtcacacagacctcattcaggaggcaaaagacgacttggcgtctcaaggatggcaggggctgcaccttggagggaactacgttgctggagtggctcttggaaagtgcatggaagcgggatataagcaggccgctgagctagccagtcacgttaaaagtctggcaacctga

SEQ ID NO: 8 - DNA, 1416 bp, Artificial Sequence
  Synthetic (Optimized codon encoding MxPPO)
atgcaccatatgccccgaacaacaggaatgaacgtcgctgtcgtgggaggcggaatatccggccttgctgtagcgcaccaccttagatccagggggaccgacgccgtcctactggaaagctccgcccgattgggcggagcagtgggtacacacgcgttggcgggttacttagtggagcaaggtccaaatagttttcttgacagggagccggcaacgcgagccctggctgctgcgcttaacttagaaggccgaataagggcagccgatccggctgcaaagagacgttatgtctacacgcgaggacgactccgtagcgttcctgccagccctcccgcctttctggcatctgatattttacccttaggtgcaaggttaagagtggccggagagttatttagcaggcgtgcgcccgaaggagtcgatgagtccctggccgcctttgggcgaaggcatttgggccatagagcgacccaagttctattagatgcagtgcagacagggatatatgccggagatgttgagcaattatccgtggccgctacgttccccatgttagtgaaaatggaacgtgaacacaggtcacttattttaggagcaattcgagcacaaaaggctcagagacaagcggcccttccagcaggaacggccccgaagctgtcaggcgcgctgagtacatttgacggcggcttgcaagtccttatcgatgccctagccgcgtcccttggggacgcagcccacgtcggagccagggtggaaggtctggctagagaagacgggggctggaggttgatcatcgaagaacacggcagacgagcggagttgtcagtggctcaagtagtgctagccgctccagcgcacgcaactgcaaagttgcttcgacccttagacgatgcactggcagccctagtggcaggtatcgcctatgcccccatagctgtagttcatctcggattcgacgccggaactttaccggcgcccgatgggttcggttttttagtgccggcggaggaacagcgaaggatgttgggtgccattcatgcttccacgactttcccgttcagggctgagggtgggcgtgtcttatactcatgtatggtggggggtgcaagacaaccaggcttggtggagcaggacgaggacgccctcgcggccttagcccgagaagaattgaaagccttggcaggcgtgacggctcgacccagtttcactagagtatttcgttggcccttgggcattcctcagtacaatcttgggcaccttgaacgtgtagctgcaatcgatgccgccctgcaacgtcttcccggactgcatcttataggaaatgcatataagggggtggggctcaacgactgtattcgtaacgcagctcagcttgcagatgcactggtagctggtaatacatcacacgcgccttag

SEQ ID NO: 9 - DNA, 30 bp, Artificial Sequence
  Synthetic (ApPPO1_XbaIF primer)
cccctctagaatggccgagtacgacgttgt

SEQ ID NO: 10 - DNA, 30 bp, Artificial Sequence
  Synthetic (ApPPO1_XhoIR primer)
ccccctcgagggttgccagacttttaacgt

SEQ ID NO: 11 - DNA, 30 bp, Artificial Sequence
  Synthetic (MxPPO_XbaIF primer)
cccctctagaatgcaccatatgccccgaac

SEQ ID NO: 12 - DNA, 30 bp, Artificial Sequence
  Synthetic (MxPPO_XhoIR primer)
ccccctcgagaggcgcgtgtgatgtattac

SEQ ID NO: 13 - DNA, 32 bp, Artificial Sequence
  Synthetic (Y422M F primer)
ctcttgtcactttatgggggggctaccaacac

SEQ ID NO: 14 - DNA, 32 bp, Artificial Sequence
  Synthetic (Y422M R primer)
cccccataaagtgacaagagcagcacctttcc

SEQ ID NO: 15 - DNA, 32 bp, Artificial Sequence
  Synthetic (Y422L F primer)
ctcttgtcactttttgggggggctaccaacac

SEQ ID NO: 16 - DNA, 32 bp, Artificial Sequence
  Synthetic (Y422L R primer)
cccccaaaaagtgacaagagcagcacctttcc

SEQ ID NO: 17 - DNA, 31 bp, Artificial Sequence
  Synthetic (Y422C F primer)
tcttgtcatgttttgggggggctaccaacac

SEQ ID NO: 18 - DNA, 32 bp, Artificial Sequence
  Synthetic (Y422C R primer)
cccccaaaacatgacaagagcagcacctttcc

SEQ ID NO: 19 - DNA, 32 bp, Artificial Sequence
  Synthetic (Y422V F primer)
ctcttgtcagtttttgggggggctaccaacac

SEQ ID NO: 20 - DNA, 31 bp, Artificial Sequence
  Synthetic (Y422V R primer)
cccccaaaaactgacaagagcagcacctttc

SEQ ID NO: 21 - DNA, 32 bp, Artificial Sequence
  Synthetic (Y422I F primer)
ctcttgtcaatttttgggggggctaccaacac

SEQ ID NO: 22 - DNA, 31 bp, Artificial Sequence
  Synthetic (Y422I R primer)
ccccaaaaattgacaagagcagcacctttcc

SEQ ID NO: 23 - DNA, 33 bp, Artificial Sequence
  Synthetic (Y422T F primer)
gtgctgctcttgtcaacctttgggggggctacc

SEQ ID NO: 24 - DNA, 33 bp, Artificial Sequence
  Synthetic (Y422T R primer)
ggtagcccccccaaaggttgacaagagcagcac

SEQ ID NO: 25 - DNA, 31 bp, Artificial Sequence
  Synthetic (A215L F primer)
gggtttacctcggcgacccggctaagttgag

SEQ ID NO: 26 - DNA, 31 bp, Artificial Sequence
  Synthetic (A215L R primer)
gtcgccgaggtaaaccccgctgcaaaacggc

SEQ ID NO: 27 - DNA, 31 bp, Artificial Sequence
  Synthetic (A215C F primer)
gggtttactgcggcgacccggctaagttgag

SEQ ID NO: 28 - DNA, 31 bp, Artificial Sequence
  Synthetic (A215C R primer)
gtcgccgcagtaaaccccgctgcaaaacggc

SEQ ID NO: 29 - DNA, 32 bp, Artificial Sequence
  Synthetic (V360M F primer)
ccctcccatggcatctgtagcattatcttacc

SEQ ID NO: 30 - DNA, 32 bp, Artificial Sequence
  Synthetic (V360M R primer)
tacagatgccatgggagggtaatagatagagc

SEQ ID NO: 31 - DNA, 34 bp, Artificial Sequence
  Synthetic (R140A F primer)
gatccgaaggcgcccgcgtacgtttattggggtg

SEQ ID NO: 32 - DNA, 34 bp, Artificial Sequence
  Synthetic (R140A R primer)
caccccaataaacgtacgcgggcgccttcggatc

SEQ ID NO: 33 - DNA, 33 bp, Artificial Sequence
  Synthetic (F209A F primer)
cgacttatagagccggcgtgcagcggggtttac

SEQ ID NO: 34 - DNA, 33 bp, Artificial Sequence
  Synthetic (F209A R primer)
gtaaaccccgctgcacgccggctctataagtcg

SEQ ID NO: 35 - DNA, 33 bp, Artificial Sequence
  Synthetic (V213C F primer)
ccgttttgcagcgggtgctacgccggcgacccg

SEQ ID NO: 36 - DNA, 33 bp, Artificial Sequence
  Synthetic (V213C R primer)
cgggtcgccggcgtagcacccgctgcaaaacgg

SEQ ID NO: 37 - DNA, 34 bp, Artificial Sequence
  Synthetic (F386V F primer)
ggtcacctagcgggcgtgggccagctacaccctc

SEQ ID NO: 38 - DNA, 34 bp, Artificial Sequence
  Synthetic (F386V R primer)
gagggtgtagctggcccacgcccgctaggtgacc

SEQ ID NO: 39 - DNA, 33 bp, Artificial Sequence
  Synthetic (L389T F primer)
gcgggctttggccagacccaccctcgtactcag

SEQ ID NO: 40 - DNA, 33 bp, Artificial Sequence
  Synthetic (L389T R primer)
ctgagtacgagggtgggtctggccaaagcccgc

SEQ ID NO: 41 - DNA, 34 bp, Artificial Sequence
  Synthetic (I402T F primer)
caccactctgggcactacctatgcctcaagctta

SEQ ID NO: 42 - DNA, 34 bp, Artificial Sequence
  Synthetic (I402T R primer)
taagcttgaggcataggtagtgcccagagtggtg

SEQ ID NO: 43 - DNA, 33 bp, Artificial Sequence
  Synthetic (V360I F primer)
ctattaccctcccatcgcatctgtagcattatc

SEQ ID NO: 44 - DNA, 33 bp, Artificial Sequence
  Synthetic (V360I R primer)
gataatgctacagatgcgatgggagggtaatag

SEQ ID NO: 45 - DNA, 33 bp, Artificial Sequence
  Synthetic (V360L F primer)
taccctcccctcgcatctgtagcattatcttac

SEQ ID NO: 46 - DNA, 30 bp, Artificial Sequence
  Synthetic (V360L R primer)
cagatgcgaggggagggtaatagatagagc

SEQ ID NO: 47 - DNA, 33 bp, Artificial Sequence
  Synthetic (S362V F primer)
taccctcccgtcgcagttgtagcattatcttac

SEQ ID NO: 48 - DNA, 33 bp, Artificial Sequence
  Synthetic (S362V R primer)
gtaagataatgctacaactgcgacgggagggta

SEQ ID NO: 49 - DNA, 35 bp, Artificial Sequence
  Synthetic (V213C+A215C F primer)
ttgcagcgggtgctactgcggcgacccggctaagt

SEQ ID NO: 50 - DNA, 35 bp, Artificial Sequence
  Synthetic (V213C+A215C R primer)
acttagccgggtcgccgcagtagcacccgctgcaa

SEQ ID NO: 51 - DNA, 35 bp, Artificial Sequence
  Synthetic (V213C+A215L F primer)
ttgcagcgggtgctaccttggcgacccggctaagt

SEQ ID NO: 52 - DNA, 35 bp, Artificial Sequence
  Synthetic (V213C+A215L R primer)
acttagccgggtcgccaaggtagcacccgctgcaa

SEQ ID NO: 53 - DNA, 31 bp, Artificial Sequence
  Synthetic (M365T F primer)
actcatgtacggtggggggtgcaagacaacc

SEQ ID NO: 54 - DNA, 32 bp, Artificial Sequence
  Synthetic (M365T R primer)
ccccaccgtacatgagtataagacacgcccac

SEQ ID NO: 55 - DNA, 32 bp, Artificial Sequence
  Synthetic (M365L F primer)
tactcatgtctggtggggggtgcaagacaacc

SEQ ID NO: 56 - DNA, 31 bp, Artificial Sequence
  Synthetic (M365L R primer)
cccaccagacatgagtataagacacgcccac

SEQ ID NO: 57 - DNA, 32 bp, Artificial Sequence
  Synthetic (M365C F primer)
tactcatgttgcgtggggggtgcaagacaacc

SEQ ID NO: 58 - DNA, 31 bp, Artificial Sequence
  Synthetic (M365C R primer)
cccccacgcaacatgagtataagacacgccc

SEQ ID NO: 59 - DNA, 32 bp, Artificial Sequence
  Synthetic (M365V F primer)
tactcatgtgtggtggggggtgcaagacaacc

SEQ ID NO: 60 - DNA, 32 bp, Artificial Sequence
  Synthetic (M365V R primer)
ccccccaccacacatgagtataagacacgccc

SEQ ID NO: 61 - DNA, 34 bp, Artificial Sequence
  Synthetic (M365I F primer)
gtcttatactcatgtatcgtggggggtgcaagac

SEQ ID NO: 62 - DNA, 34 bp, Artificial Sequence
  Synthetic (M365I R primer)
gtcttgcaccccccacgatacatgagtataagac

SEQ ID NO: 63 - DNA, 32 bp, Artificial Sequence
  Synthetic (R95A F primer)
gcaaagagagcttatgtctacacgcgaggacg

SEQ ID NO: 64 - DNA, 33 bp, Artificial Sequence
  Synthetic (R95A R primer)
gtagacataagctctctttgcagccggatcggc

SEQ ID NO: 65 - DNA, 31 bp, Artificial Sequence
  Synthetic (V164A F primer)
tagatgcagcgcagacagggatatatgccgg

SEQ ID NO: 66 - DNA, 30 bp, Artificial Sequence
  Synthetic (V164A R primer)
ctgtctgcgctgcatctaatagaacttggg

SEQ ID NO: 67 - DNA, 31 bp, Artificial Sequence
  Synthetic (I168C F primer)
cagacagggtgctatgccggagatgttgagc

SEQ ID NO: 68 - DNA, 33 bp, Artificial Sequence
  Synthetic (I168C R primer)
tccggcatagcaccctgtctgcactgcatctaa

SEQ ID NO: 69 - DNA, 32 bp, Artificial Sequence
  Synthetic (A170C F primer)
gggatatattgcggagatgttgagcaattatc

SEQ ID NO: 70 - DNA, 32 bp, Artificial Sequence
  Synthetic (A170C R primer)
acatctccgcaatatatccctgtctgcactgc

SEQ ID NO: 71 - DNA, 32 bp, Artificial Sequence
  Synthetic (A170L F primer)
gggatatatctcggagatgttgagcaattatc

SEQ ID NO: 72 - DNA, 32 bp, Artificial Sequence
  Synthetic (A170L R primer)
acatctccgagatatatccctgtctgcactgc

SEQ ID NO: 73 - DNA, 31 bp, Artificial Sequence
  Synthetic (I311M F primer)
tgcccccatggctgtagttcatctcggattc

SEQ ID NO: 74 - DNA, 30 bp, Artificial Sequence
  Synthetic (I311M R primer)
aactacagccatgggggcataggcgatacc

SEQ ID NO: 75 - DNA, 32 bp, Artificial Sequence
  Synthetic (F329V F primer)
cgatggggtcggttttttagtgccggcggagg

SEQ ID NO: 76 - DNA, 31 bp, Artificial Sequence
  Synthetic (F329V R primer)
aaaaaaccgaccccatcgggcgccggtaaag

SEQ ID NO: 77 - DNA, 30 bp, Artificial Sequence
  Synthetic (L332T F primer)
ttcggttttacagtgccggcggaggaacag

SEQ ID NO: 78 - DNA, 31 bp, Artificial Sequence
  Synthetic (L332T R primer)
ccggcactgtaaaaccgaacccatcgggcgc

SEQ ID NO: 79 - DNA, 31 bp, Artificial Sequence
  Synthetic (I345T F primer)
gggtgccactcatgcttccacgactttcccg

SEQ ID NO: 80 - DNA, 32 bp, Artificial Sequence
  Synthetic (I345T R primer)
gaagcatgagtggcacccaacatccttcgctg

SEQ ID NO: 81 - DNA, 33 bp, Artificial Sequence
  Synthetic (I168C+A170C F primer)
gtgcagacagggtgctattgcggagatgttgag

SEQ ID NO: 82 - DNA, 33 bp, Artificial Sequence
  Synthetic (I168C+A170C R primer)
ctcaacatctccgcaatagcaccctgtctgcac

SEQ ID NO: 83 - DNA, 30 bp, Artificial Sequence
  Synthetic (ApPPO1_BamHIF primer)
ccccggatccatggccgagtacgacgttgt

SEQ ID NO: 84 - DNA, 33 bp, Artificial Sequence
  Synthetic (ApPPO1_SalIR primer)
ccccgtcgactcaggttgccagacttttaacgt

SEQ ID NO: 85 - DNA, 30 bp, Artificial Sequence
  Synthetic (MxPPO_BamHIF primer)
ccccggatccatgcaccatatgccccgaac

SEQ ID NO: 86 - DNA, 33 bp, Artificial Sequence
  Synthetic (MxPPO_SalIR primer)
ccccgtcgactcaaggcgcgtgtgatgtattac

SEQ ID NO: 87 - DNA, 1614 bp, Artificial Sequence
  Synthetic (Gene for AtPPO1 of Arabidopsis thaliana)
atggagttatctcttctccgtccgacgactcaatcgcttcttccgtcgttttcgaagcccaatctccgattaaatgtttataagcctcttagactccgttgttcagtggccggtggaccaaccgtcggatcttcaaaaatcgaaggcggaggaggcaccaccatcacgacggattgtgtgattgtcggcggaggtattagtggtctttgcatcgctcaggcgcttgctactaagcatcctgatgctgctccgaatttaattgtgaccgaggctaaggatcgtgttggaggcaacattatcactcgtgaagagaatggttttctctgggaagaaggtcccaatagttttcaaccgtctgatcctatgctcactatggtggtagatagtggtttgaaggatgatttggtgttgggagatcctactgcgccaaggtttgtgttgtggaatgggaaattgaggccggttccatcgaagctaacagacttaccgttctttgatttgatgagtattggtgggaagattagagctggttttggtgcacttggcattcgaccgtcacctccaggtcgtgaagaatctgtggaggagtttgtacggcgtaacctcggtgatgaggtttttgagcgcctgattgaaccgttttgttcaggtgtttatgctggtgatccttcaaaactgagcatgaaagcagcgtttgggaaggtttggaaactagagcaaaatggtggaagcataataggtggtacttttaaggcaattcaggagaggaaaaacgctcccaaggcagaacgagacccgcgcctgccaaaaccacagggccaaacagttggttctttcaggaagggacttcgaatgttgccagaagcaatatctgcaagattaggtagcaaagttaagttgtcttggaagctctcaggtatcactaagctggagagcggaggatacaacttaacatatgagactccagatggtttagtttccgtgcagagcaaaagtgttgtaatgacggtgccatctcatgttgcaagtggtctcttgcgccctctttctgaatctgctgcaaatgcactctcaaaactatattacccaccagttgcagcagtatctatctcgtacccgaaagaagcaatccgaacagaatgtttgatagatggtgaactaaagggttttgggcaattgcatccacgcacgcaaggagttgaaacattaggaactatctacagctcctcactctttccaaatcgcgcaccgcccggaagaattttgctgttgaactacattggcgggtctacaaacaccggaattctgtccaagtctgaaggtgagttagtggaagcagttgacagagatttgaggaaaatgctaattaagcctaattcgaccgatccacttaaattaggagttagggtatggcctcaagccattcctcagtttctagttggtcactttgatatccttgacacggctaaatcatctctaacgtcttcgggctacgaagggctatttttgggtggcaattacgtcgctggtgtagccttaggccggtgtgtagaaggcgcatatgaaaccgcgattgaggtcaacaacttcatgtcacggtacgcttacaagtaa